(19) United States
(12) Patent Application Publication
(10) Pub. No.: US 2024/0327841 A1
(43) Pub. Date: Oct. 3, 2024

(54) COMPOSITIONS COMPRISING DOPED SILICON PARTICLES, AND RELATED METHODS

(71) Applicant: SISAF LTD, Guildford (GB)

(72) Inventors: Roghieh Suzanne SAFFIE-SIEBERT, Guildford (GB); Mukhtar AHMED, Guildford (GB); Flavia SUTERA, Guildford (GB)

(73) Assignee: SISAF LTD, Guildford (GB)

(21) Appl. No.: 18/580,594

(22) PCT Filed: Jul. 22, 2022
(86) PCT No.: PCT/GB2022/051937

§ 371 Date: Jan. 18, 2024

(30) Foreign Application Priority Data
Jul. 23, 2021 (GB) 2110644.8

(51) Int. Cl. C12N 15/113; A61K 9/14; A61K 31/7105

(52) U.S. Cl. CPC C12N 15/113; A61K 9/143; A61K 9/145; A61K 31/7105; C12N 2310/14

(57) ABSTRACT

A pharmaceutical composition comprising particles comprising hydrolysable doped silicon and one or more lipids complexed with an active pharmaceutical ingredient, wherein the particles are doped at a level of at least 1×10¹⁶ dopant atoms per cm³. Also related products, methods, and medical uses.

### FIELD OF INVENTION

The invention relates to improved particles for use in compositions comprising nucleic acids and/or other pharmaceutically-active compounds, and related products and methods. Such methods, products and compositions are especially useful for, although not limited to, the delivery of nucleic acid in gene therapy and vaccine compositions. Particles of the invention comprise silicon which has been doped with one or more further elements. The invention also relates to pharmaceutical compositions comprising particles of the invention, and to related methods and uses.

### BACKGROUND

There is a need for improvements in delivery vectors and vehicles for active pharmaceutical agents. This is required for advances in biomedical research to be fully translated into effective, safe and cost-effective treatments.

As an illustrative example, nucleic acids such as RNA have been proposed as therapeutic agents. Small interfering RNA (siRNA) has been proposed for use in gene therapy. Gene delivery for therapy or other purposes is well-known, particularly for the treatment of diseases such as cystic fibrosis and certain cancers, and mRNA has been recently used in effective vaccines against SARS-CoV-2. As used herein, the term “gene therapy” refers to the delivery into a cell of a gene or part of a gene to correct some deficiency. In the present specification, the term “nucleic acid therapy” is used also to refer to any introduction of nucleic acid material into target cells, and includes gene vaccination. Also, the term “nucleic acid delivery” may encompass the in vitro production of commercially-useful proteins in so-called cell factories.

Delivery systems for delivering nucleic acids to cells fall into three broad classes, namely those that involve direct injection of naked nucleic acid: those that make use of viruses or genetically modified viruses: and those that make use of non-viral delivery agents. Each has its advantages and disadvantages. Although viruses as delivery agents have the advantages of high efficiency and high cell selectivity, they have the disadvantages of toxicity, production of inflammatory responses and difficulty in delivering large nucleic acid fragments. Accordingly, an mRNA vaccine may comprise injectable naked mRNA or non-viral delivery systems such as lipid nanoparticle vectors. Unfortunately, lower transfection efficiencies have been noted with non-viral delivery systems, mRNA also has well-known stability problems.

Non-viral gene delivery systems are based on the compaction of genetic material into nanometric particles by electrostatic interaction between the negatively charged phosphate backbone of the nucleic acid and cationic lipids and, optionally, peptides or other compounds (Erbacher, P, et al., Gene Therapy, 1999, 6, 138-145). The mechanism of payload delivery by lipid nanoparticle non-viral vectors is proposed to involve endocytosis of intact complexes, in which complexes formed between the nucleic acid and the lipid become attached to the cell surface, then enter the cell by endocytosis. The complex then remains localised within a vesicle or endosome for some time and the nucleic acid component is then released into the cytoplasm. Production of a protein encoded by the nucleic acid may then take place or the expression of another gene modification.

The components of a non-viral delivery system associate electrostatically to form a vector complex. The lipid component shields both the nucleic acid and, to a degree, any peptide component(s) from degradation, endosomal or otherwise. Cationic lipids for such a use were developed by Felgner in the late 1980s and reported in Proc. Natl. Acad. Sci. USA 84, 7413-7417, 1987 and in U.S. Pat. No. 5,264,618. Felgner developed the now commercially-available cationic liposome known by the trademark “Lipofectin”. The “Lipofectin” liposome is a spherical vesicle having a lipid bilayer of the cationic lipid DOTMA (2,3-dioleyloxypropyl-1-trimethyl ammonium) and the neutral phospholipid lipid DOPE (phosphatidyl ethanolamine or 1,2-dioleoyl-sn-glycero-3-phosphoethanolamine) in a 1:1 ratio. Various other cationic liposome formulations have since been devised, most of which combine a synthetic cationic lipid and a neutral lipid. In addition to the DOTMA analogues, there may be mentioned complex alkylamine/alkylamides, cholesterol derivatives, such as DC-Chol, and synthetic derivatives of dipalmitol, phosphatidyl-ethanolamine, glutamate, imidazole and phosphonate. However, cationic vector systems vary enormously in their transfection efficiencies in the presence of serum, which clearly impacts on their potential uses for in vivo gene therapy and vaccination. Ionizable lipids, such as positively charged lipids, are useful transfection agents because their positive charges tend to allow them to complex with a negatively charged nucleic acid. It is understood that different lipids have different levels of positive charge. Unfortunately, there are many lipids which are insufficiently positively charged to allow complexing of nucleic acids sufficient to adequately protect the nucleic acid from degradation, for example during a period of extended storage. Conversely, a lipid which is very highly positively charged, may be highly efficient at binding nucleic acid, and may therefore be a promising candidate for use in protecting a nucleic acid during long term storage, but may have toxicity issues which prevent its clinical use. For example, polyethylenimines (PEIs) are highly cationic and used as an efficient in vitro transfection lipid. They are however toxic which precludes their use in clinical treatments. Lipid transfection agents may also degrade (or “age”) during storage which reduces their ability to protect nucleic acid to which they are complexed and may require the use of lipid in excess amounts to mitigate expected losses of activity with time.

There exist a surprisingly small number of lipid transfection agents suitable and approved for clinical use which have both an acceptable toxicity profile and which cause efficient transfection. 1,2-dioleoyl-3-trimethylammonium-propane (DOTAP) is currently a favoured transfection reagent in many applications (including Pfizer's SARS-CoV-2 mRNA vaccine) but it has supply issues and few alternatives are available.

Turning to the optional peptide component of a non-viral delivery system, referred to above: a peptide for optional use alongside lipids in a transfection complex typically has two functionalities: a “head group” containing a cell surface receptor-(for example, integrin-) recognition sequence and a “tail” that can bind nucleic acid (for example, mRNA) non-covalently. Such a peptide component can be designed, to a certain degree, to be cell-type specific or cell-surface receptor specific. Specificity results from targeting to the cell-surface receptors. A degree of integrin specificity, for example, can confer a degree of cell specificity to the complex. Transfection efficiencies comparable to some adenoviral vectors can be achieved (Jenkins et al. Gene Therapy 7, 393-400, 2000). However, there remains a need for compositions having the ability to target a specific cell, or a specific tissue, without reliance on such peptides.

The present invention seeks to increase the efficiency of pharmaceutical compositions for the delivery of an active pharmaceutical ingredient, in particular lipid transfection agents, in order to achieve one or more of the following advantages:
1) an increase in the efficiency of the ability of a lipid to protect a nucleic acid from degradation during storage;
2) an increase in transfection efficiency;
3) an increase in the stability of a lipid used to protect a nucleic acid from degradation during storage;
4) the ability to use lower levels of lipid, and particularly lower levels of cationic lipid such as DOTMA or DOTAP, in transfection compositions whilst still maintaining adequate transfection ability and/or good storage stability;
5) the ability to use a wider range of lipids in transfection compositions whilst still maintaining adequate transfection ability and/or good storage stability; and
6) more targeted delivery of an active pharmaceutical ingredient, such as a nucleic acid, to a particular type of tissue, or to a particular type of cell.

The non-viral delivery of messenger RNA (mRNA) to cells has so far been particularly problematic and limited by the lack of an efficient vector. Attempts to deliver mRNA using known non-viral vehicles may result in sub-optimal levels of protein expression, for example due to poor targeting of the mRNA to a specific tissue or to a specific cell. Furthermore, known non-viral vehicles have poor storage stability when packaged with mRNA. Overcoming the lipid bilayer to deliver RNA into cells has remained a major obstacle for the widespread development of RNA therapeutics.

Therefore, there is a need for vectors that are specifically tailored to the delivery of mRNA that deliver high levels of the mRNA to cells, optionally with specificity for a particular type of tissue or for a particular type of cell, and lead to good levels of protein expression. There is also a need for compositions tailored to the delivery of mRNA that have good stability upon storage, in particular mRNA delivery complexes that retain their structure and functionality upon storage at moderate temperatures. Similar considerations apply to the delivery of siRNA therapeutics.

A number of mRNA vaccines against SARS-CoV-2, including the Pfizer BioNTech vaccine BNT162b2 (“Comirnaty”), and the Moderna CX-024414 vaccine, require ultra-cold-chain storage and transport. This limits accessibility to the vaccine for low-income countries and adds cost and logistical complexity in all markets. It would be advantageous if vaccines can be stored and transported at standard refrigerator temperature (about 4° C.) or room temperature (about 20° C.). It would also be of benefit if vaccines could tolerate higher temperatures (for example, 30, 40 or 50° C.) for storage, or at the very least in the short-term during transport and distribution. Specifically, it would be useful if existing mRNA vaccine formulations comprising mRNA and a lipid could be modified to increase their transfection efficiency. This would have the benefit of allowing a lower dose to be used, potentially reducing side-effects and increasing the total number of doses available. It would also be useful if existing mRNA vaccine formulations comprising mRNA and a lipid could be modified to improve their storage stability thereby permitting distribution and storage at a higher temperature and/or for longer time periods. It would also be useful if existing mRNA vaccine formulations could be modified to require lower levels of lipid (in particular lower levels of cationic lipids, such as DOTAP) and/or to function with a wider range of lipids in order to reduce pressure on availability of specific lipids, particularly cationic lipids, such as DOTAP.

Maintaining mRNA stability in an injectable composition, for example an mRNA vaccine composition, by means of low temperatures, as well as bringing logistical challenges, also has the technical limitation that the mRNA must be defrosted before injection, and that after injection it must remain stable at the higher temperatures of the body for long enough to show sufficient biological activity. This may require stability to be maintained during translocation around the body and/or escape from the endosomal compartment. Stability in vivo must also be maintained for long enough for sufficient translation into protein to take place.

mRNA is vulnerable to enzymatic and chemical degradation. Enzymes able to degrade RNA, such as mRNA, are present in the biological culture systems used to produce mRNA and are difficult to remove completely. Enzymatic activity may be slowed by low temperature and/or by lyophilization of the RNA, but each of those solutions come with disadvantages.

Encapsulation with lipid has been used in the prior art to protect RNA (for example mRNA for gene therapy or vaccination) from degradation. Whilst this approach can work, it uses relatively large amounts of specific lipids which may be expensive and/or in short supply and it presents problems of the lipid itself degrading over time and thus losing its protective qualities. There is also a need for improved excipients for increasing the stability of multiple components of a composition comprising nucleic acids and lipid.

Although the compositions and methods of the invention are currently thought to be most promising in improving pharmaceutical compositions comprising nucleic acids and lipid, they are also suitable for the stabilisation and protection from degradation of non-nucleic acid active ingredients, including small organic compounds and peptides (for example peptide antigens).

### BRIEF DESCRIPTION OF INVENTION

The present invention is based on the realisation that elemental silicon which has been doped, particularly which has been doped at a level of at least 1×10¹⁵, especially at least 1×10¹⁶ dopant atoms per cm³, especially with boron, is useful in stabilising an active pharmaceutical ingredient, particularly a nucleic acid, within a composition, such as a transfection composition, comprising one or more lipids. Such compositions show enhanced transfection efficiency, enhanced tissue or cell targeting ability, a decreased reliance on cationic lipids, and/or enhanced storage stability. Doped silicon can stabilise both the active pharmaceutical ingredient (particularly a nucleic acid) itself and also stabilise the one or more lipids, so that the one or more lipids can retain, for a longer period of time, properties which allow it to protect the active pharmaceutical ingredient, particularly a nucleic acid. Stabilisation of the lipid and/or nucleic acid can enable the composition to be stored, without problematic degradation, for longer and/or at higher temperature than has been possible with conventional compositions having active pharmaceutical ingredients that are nucleic acids (e.g., at room temperature, or at 4° C.) yet which do not comprise hydrolysable doped silicon particles. It can also allow the composition to be more efficient in transfection of cells, for example by providing improved targeting action to a specific tissue type and/or a specific cell type, and in the subsequent provision of one or more therapeutic effects by the active pharmaceutical ingredient, particularly a nucleic acid. Although some of the challenges of stability and efficient delivery are especially acute with regard to nucleic acid (e.g. mRNA) therapeutics, it has further been found that the stabilising and protective properties of compositions comprising hydrolysable doped silicon particles and one or more lipids, are applicable to other non-nucleic acid active pharmaceutical ingredients, such as peptides, proteins and small molecules. Doping of hydrolysable silicon, may enable different amphiphilic molecules, with various functionalities, to be combined in a matrix crystal structure of the hydroly sable silicon material; thus, hydrolysable doped silicon not only counteracts the instability of organic molecules, such as lipids, but also enhances the stability of active pharmaceutical ingredient, particularly nucleic acid (e.g. mRNA) molecules. It is thought that the advantages of the present invention derive not only from the binding or loading of organic compounds, which may for example occur within pores in the hydrolysable doped silicon particles; but also the intentionally introduced impurities (dopants), which may be used as part of a structural scaffold for secondary binding of organic materials, such as the one or more lipids.

According to a first aspect of the invention there is provided a pharmaceutical composition comprising particles comprising hydrolysable doped silicon and at least one lipid complexed with an active pharmaceutical ingredient. Optionally, the particles are doped at a level of at least 1×10¹⁶ dopant atoms per cm³.

According to a second aspect of the invention there is provided the use of particles comprising hydrolysable doped silicon, optionally hydrolysable boron doped silicon, to enhance the efficiency of a pharmaceutical composition comprising an active pharmaceutical ingredient. Again, optionally, the particles are doped at a level of at least 1×10¹⁶ dopant atoms per cm³.

According to a third aspect of the invention there is provided a pharmaceutical composition according to the first aspect of the invention for use as a medicament.

According to a fourth aspect of the invention there is provided the use of a pharmaceutical composition according to the first aspect of the invention in the manufacture of a medicament, for example a vaccine.

According to a fifth aspect of the invention there is provided a method of treating or preventing a disease or disorder in a subject comprising administering to a subject in need thereof a pharmaceutical composition according to the first aspect of the invention.

According to a sixth aspect of the invention there is provided a method of providing a vaccine to a subject in need thereof comprising administering to said subject a pharmaceutical composition according to the first aspect of the invention.

According to a seventh aspect of the invention there is provided a method of increasing the storage stability of an active pharmaceutical ingredient, for example a nucleic acid, for example a mRNA or saRNA or shRNA or siRNA, the method comprising contacting the nucleic acid with hydrolysable doped silicon particles and one or more lipid.

According to an eighth aspect of the invention there is provided a pharmaceutical composition according to the first aspect of the invention, for use in targeting an active pharmaceutical ingredient to a cell or tissue.

According to a ninth aspect of the invention there is provided a pharmaceutical composition according to the first aspect of the invention, for use in the manufacture of a medicament for the targeting of an active pharmaceutical ingredient to a cell or tissue.

According to a tenth aspect of the invention there is provided a method of targeting an active pharmaceutical ingredient to a cell or tissue, comprising administering to a subject in need thereof a pharmaceutical composition according to the first aspect of the invention.

### DESCRIPTION OF THE DRAWINGS

FIG. 1 shows silicon after boron doping.

FIG. 2 shows the boron doped silicon of FIG. 1 after grinding to a powder.

FIG. 3 shows ClCn7G213R expression in mouse PMBCs.

FIG. 4 shows bone expression of ClCn7G213R.

FIG. 5 shows CTX-blood test results.

FIG. 6 shows gel electrophoresis images of the Biocouriers loaded with pDNA shortly after preparation and 6 h after storage at room temperature (RT).

FIG. 7 shows gel electrophoresis images of the Biocouriers loaded with pDNA 24 and 48 h after storage at room temperature (RT).

FIG. 8 shows gel electrophoresis images of the Biocouriers loaded with pDNA 72 h and 8 days after storage at room temperature (RT).

FIG. 9 shows gel electrophoresis images of the Biocouriers loaded with pDNA shortly after preparation and 6 h after storage at 4° C.

FIG. 10 shows gel electrophoresis images of the Biocouriers loaded with pDNA 24 and 48 h after storage at 4° C.

FIG. 11 shows gel electrophoresis images of the Biocouriers loaded with pDNA 72 h and 8 days after storage at 4° C.

FIG. 12 shows an agarose gel retardation assay of the RNA from baker's yeast loaded onto SIS0012 at different concentrations and volume ratios. Naked RNA was used as control. In the first 3 columns of images, the box around some of the loading wells indicates control samples. In the final column of images lanes 2, 3, 4 contain SIS0113 at X10 dilution, V/V: 2.5, lanes 5, 6, 7 contain SIS0113 at X5 dilution, V/V: 2.5, lanes 8, 9, 10 contain SIS0113 at X5 dilution, V/V: 5,

FIG. 13 shows an agarose gel retardation assay of the DNA from Herring testes loaded onto SIS0012 at the selected concentration and volume ratio at different time points (0-5 h) following storage at different temperatures (room temperature (RT), 4° C. and −20° C.). Naked DNA was used as control. In the top left image the box around some of the loading wells indicates control samples. In all other images lanes 2, 3, 4 hold samples stored at room temperature, lanes 5, 6,7 samples stored at 4° C., lanes 8, 9, 10 samples stored at 20° C.

FIG. 14 shows an agarose gel retardation assay of the DNA from Herring testes loaded onto SIS0012 at the selected concentration and volume ratio at different time points (24-72 h) following storage at different temperatures (room temperature (RT), 4° C. and −20° C.). Naked DNA was used as a control. In the left and middle columns of images lanes 2, 3, 4 hold samples stored at room temperature, lanes 5, 6,7 samples stored at 4° C., lanes 8, 9, 10 samples stored at 20° C. In the two right-most images, lanes 2, 3, 4 hold samples stored at room temperature, lanes 5, 6,7 samples stored at 4° C., lanes 8, 9, samples stored at 20° C., lane 10 control samples.

FIG. 15 shows an agarose gel retardation assay of ADO-siRNA loaded onto SIS0012 at the selected concentration and volume ratio at different time points (0-6 h) following storage at different temperatures (room temperature (RT), 4° C. and −20° C.). Naked siRNA was used as a control (lane 1 in all images). In all images lanes 2, 3, 4 hold samples stored at room temperature, lanes 5, 6,7 samples stored at 4° C., lanes 8, 9, 10 samples stored at 20° C.

FIG. 16 shows an agarose gel retardation assay of ADO-siRNA loaded onto SIS0012 at the selected concentration and volume ratio at different time points (24-120 h) following storage at different temperatures (room temperature (RT), 4° C. and −20° C.). Naked siRNA was used as a control (lane 1 in all images). In all images lanes 2, 3, 4 hold samples stored at room temperature, lanes 5, 6,7 samples stored at 4° C., lanes 8, 9, 10 samples stored at 20° C.

FIG. 17 shows an agarose gel retardation assay of ADO-siRNA loaded onto SIS0012 without silicon nanoparticles at the selected concentration and volume ratio at different time points (0-6 h) following storage in different temperatures (room temperature (RT) and 4° C. Naked siRNA was used as a control. In the 6 right-most images lanes 5, 6, 7 hold samples stored at room temperature, lanes 8, 9, 10 hold samples stored at 4° C.

FIG. 18 shows an agarose gel retardation assay of ADO-siRNA loaded onto SIS0012 without silicon nanoparticles at the selected concentration and volume ratio at different time points (24-120 h) following storage at room temperature (RT) or 4° C. Naked siRNA was used as a control (lane 1 in all images). In all images lanes 5, 6, 7 hold samples stored at room temperature, lanes 8, 9, 10 hold samples stored at 4° C.

FIG. 19 shows an agarose gel retardation assay of ADO-siRNA loaded onto SIS0013 at the selected concentration and volume ratio at different time points (0-6 h) following storage at room temperature. Naked siRNA was used as control.

FIG. 20 shows an agarose gel retardation assay of ADO-siRNA loaded onto SIS0013 at the selected concentration and volume ratio at different time points (24-120 h) following storage at room temperature. Naked siRNA was used as control.

FIG. 21 shows an agarose gel retardation assay of ADO-siRNA loaded onto SIS0013 at the selected concentration and volume ratio at different time points (2-120 h) following storage at 4° C. Naked siRNA was used as a control. In the right-most 2 images, lanes 2, 3, 4 are labelled “72 h”. In the images immediately to the left of those, lanes 2, 3, 4 are labelled “24 h”, and lanes 5, 6, 7 are labelled “48 h”.

FIG. 22 shows a standard curve of UV-Vis absorbance at 405 nm as a measure of enzyme activity, for alkaline phosphatase at varying concentrations.

FIG. 23 shows curves of UV-Vis absorbance at 405 nm as a measure of enzyme activity, for free alkaline phosphatase, alkaline phosphatase loaded onto SIS0012, and alkaline phosphatase loaded onto SIS0013, following incubation at 50° C.

FIG. 24 shows gel electrophoresis images of SIS0012 and SIS0013 with NAD, TYR and QUE, when loaded with siRNA.

FIG. 25 shows a gel electrophoresis image of the DPPC/PAL-KTTKS-DOPE formulations of Example 6, when loaded with siRNA.

FIG. 26 shows a further gel electrophoresis image of DPPC/PAL-KTTKS-DOPE formulations of Example 6, when loaded with siRNA.

FIG. 27 shows a further gel electrophoresis image of DPPC/PAL-KTTKS-DOPE formulations of Example 6, when loaded with mRNA.

### DETAILED DESCRIPTION

The particles of the composition of all aspects of the invention comprise hydrolysable doped silicon. The silicon is doped: advantageously, the particles are doped at a level of at least 1×10¹⁵, especially at least 1×10¹⁶ dopant atoms per cm³ (for example, at least 1×10¹⁷, 1×10¹⁸, 1×10¹⁹, or 1×10²⁰ dopant atoms per cm³). The silicon may be n-doped or p-doped. All aspects of the invention include embodiments wherein the silicon is doped with one or more elements selected from Mg, P, Cu, Ga, Al, In, Bi, Ge, Li, Xe, N, Au, Pt.

Most preferably the dopant is a p-dopant: preferably the dopant comprises boron. Thus, most preferably, the dopant is boron. P-doped silicon may be especially suitable for stabilising negatively charged nucleic acid and other negatively charged pharmaceutically active ingredients. In this way, optionally, doping of hydrolysable silicon particles according to all aspects of the present invention may enable the use of less and potentially no cationic lipid, such as DOTMA or DOTAP, compared to conventional transfection compositions which do not comprise hydrolysable doped silicon, whilst still maintaining adequate transfection ability (for example, good tissue targeting ability or good cell targeting ability) and/or good storage stability.

N-doped silicon may be especially useful in stabilising positively charged pharmaceutically active ingredients and also in protecting lipids, for example positively charged lipids, from degradation which will indirectly increase the stabilization and protection of the active pharmaceutical ingredient, for example the nucleic acid.

In embodiments of the various aspects of the invention wherein the active pharmaceutical ingredient is a nucleic acid, the pharmaceutical composition optionally further comprises a polycationic nucleic acid-binding component. The term “polycationic nucleic acid-binding component” is well known in the art and refers to polymers having at least 3 repeats of cationic amino acid residues or other cationic unit bearing positively charged groups, such polymers being capable of complexion with a nucleic acid under physiological conditions. An example of a nucleic acid-binding polycationic molecule is an oligopeptide comprising one or more cationic amino acids. Such an oligopeptide may, for example, be an oligo-lysine molecule, an oligo-histidine molecule, an oligo-arginine molecule, an oligo-ornithine molecule, an oligo diaminopropionic acid molecule, or an oligo-diaminobutyric acid molecule, or a combined oligomer comprising or consisting of any combination of histidine, arginine, lysine, ornithine diaminopropionic acid, and diaminobutyric acid residues. Further examples of polycationic components include dendrimers and polyethylenimine.

### PARTICLES COMPRISING HYDROLYSABLE SILICON

According to all aspects of the invention, the doped silicon particles may be pure doped silicon, or another hydrolysable doped silicon-containing material. If they are not pure doped silicon, they contain at least 50% by weight silicon, i.e. they comprise at least 50% by weight silicon atoms based on the total mass of atoms in the particles. For example, the silicon particles may contain at least 60, 70, 80, 90 or 95% silicon. The silicon particles preferably show a rate of hydrolysis, for example in PBS buffer at room temperature, of at least 10% of the rate of hydrolysis of pure silicon particles of the same dimensions. Assays for hydrolysis of silicon-containing material are widely known in the art (see, for example, WO2011/001456, incorporated by reference herein). Although particles of the invention may contain some silica, silica is not hydrolysable silicon and at least half of the silicon atoms in the particles are in the form of elemental silicon (or doped elemental silicon).

According to all aspects of the invention, the particles comprising hydrolysable doped silicon may be nanoparticles. Nanoparticles have a nominal diameter of between 5 and 400 nm, for example 50 to 350 nm, for example 80 to 310 nm, for example 100 to 250 nm, for example 120 to 240 nm, for example 150 to 220 nm, for example about 200 nm. They may be made of either pure doped silicon or a hydrolysable doped silicon-containing material. They are preferably porous, more preferably mesoporous. The nominal diameter referred to above, may refer to the mean diameter and at least 90% of total mass of particles in a sample of particles may fall within the size range specified. Particles comprising hydrolysable doped silicon can be made porous by standard techniques such as contacting the particles with a hydrofluoric acid (HF)/ethanol mixture and applying a current. By varying the HF concentration and the current density and time of exposure, the density of pores and their size can be controlled and can be monitored by scanning electron micrography and/or nitrogen adsorption desorption volumetric isothermic measurement.

In all aspects of the invention, it is preferred that the particles are porous. If the particles are porous, their total surface area will be increased by virtue of their porosity. For example the surface area may be increased by at least 50% or at least 100% over the surface area of a corresponding non-porous particle. In many circumstances porous particles in accordance with all aspects of the invention will in reality have a much greater increase in total surface area by virtue of their porosity. Preferably, the particles are mesoporous.

According to certain embodiments the porosity is at least 30, 40, 50 or 60%. This means that, respectively, 30, 40, 50 or 60% of the particle volume in pore space. Preferred pore diameters range from 1 nm to 50 nm, for example from 5 nm to 25 nm.

### Doping

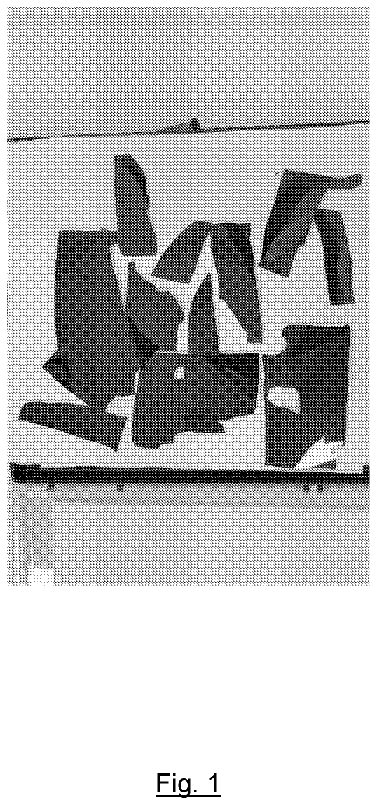

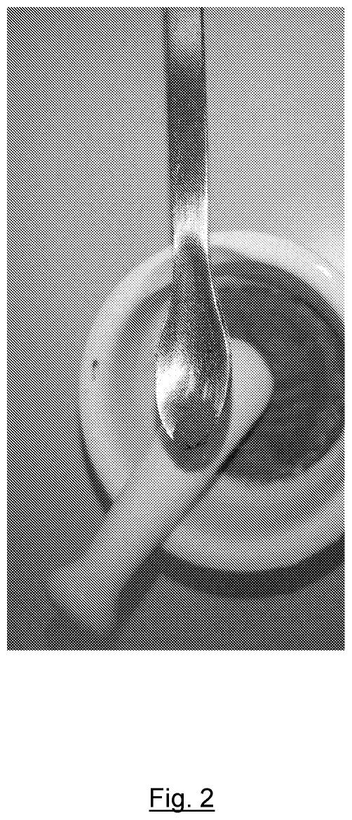

All aspects of the present invention concern doped silicon containing material. The manufacture of doped silicon is well understood in the semiconductor industry and includes ion implantation and diffusion methods. Doped silicon is therefore readily available. Alternatively, silicon can be doped by using a diffusion method to increase the amount of dopant present in the silicon. As an example of a diffusion method, silicon powder and a doping reagent (for example B₂O₃ for boron doping) is placed in a bowl which is mixed and placed under an N₂ atmosphere and subjected to a temperature of between 1050° C. and 1175° C. for a few minutes to allow the dopant (for example boron) to diffuse into the silicon. FIGS. 1 and 2 show boron doped silicon produced by this method.

In certain embodiments, doping of the silicon is heavy doping, which is understood to mean doping of at least 1×10¹⁵ dopant atoms per cm³. In some preferred embodiments, dopant is present at levels of at least 1×10¹⁶ dopant atoms per cm³. Thus, in particularly preferred embodiments, boron is present at levels of at least 1×10¹⁶ boron atoms per cm³.

For example, there may be at least 1×10¹⁷ dopant atoms per cm³, at least 1×10¹⁸ dopant atoms per cm^{3,} or at least 1×10¹⁹ dopant atoms per cm³.

Optionally, there may be up to 1×10²⁰ dopant atoms per cm³. For example, there may be boron present at levels of at least 1×10¹⁶ boron atoms per cm³ and up to 1×10²⁰ boron atoms per cm³.

When boron is used as the dopant, as is preferred, doping levels of 1×10¹⁵ dopant atoms per cm³, and 1×10²⁰ dopant atoms per cm³ correspond, respectively, to a resistivity of 13.6 ohm-cm, and 1.3 mohm-cm. The various aspects of the invention in which boron is the preferred dopant, do not exclude silicon which in addition to being doped, for example heavily doped with boron, is also doped with other elements. According to preferred embodiments of all aspects of the invention, the majority dopant is boron.

### Lipids

Lipids, in the present technical field, are generally understood to include fatty acids and fatty acid derivatives, glycerolipids, glycerophospholipids, sphingolipids, saccharolipids and polyketides.

As used in the present application, the term “lipid” may also cover lipidated oligopeptide (a term used interchangeably herein with the term lipopeptide) wherein a short peptide sequence (such as a peptide sequence having 3 to 20 amino acid residues, such as 5 to 15 amino acid residues, especially 3, 4, or 5 amino acid residues, and most especially 5 amino acid residues) is conjugated to one or more fatty acid chains (especially a fatty acid chain having a 10 to 24 carbon chain length, preferably, a 12 to 18 carbon chain length: for example a 14, 15 or 16 carbon chain length: for instance, the peptide moiety may optionally be lipidated with a palmitoyl, cetyl or myristoyl moiety).

A lipidated oligopeptide may optionally be a lipdated tetrapeptide, lipidated pentapeptide or lipidated hexapeptide. Preferably, the amino acid residues include at least one amino acid residue (for example, 2 or 3 amino acid residues) that is cationic at a pH of 7.4 (physiological pH), such as lysine or arginine. For example, the lipidated oligopeptide may include one or more (for example 2) lysine resides. Thus, a particular example is palmitoyl-pentapeptide-4 (CAS number 214047-00-4; abbreviated as PAL-KTTKS):

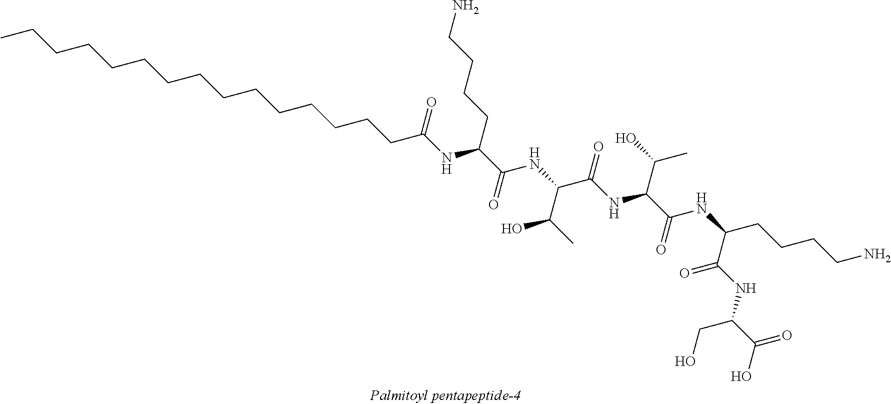 [SMILES: CCCCCCCCCCCCCCCC(=O)N[C@@H](CCCCN)C(=O)N[C@H](C(=O)N[C@H](C(=O)N[C@@H](CCCCN)C(=O)N[C@@H](CO)C(=O)O)[C@@H](C)O)[C@@H](C)O]

Thus, in certain preferred embodiments according to all aspects of the invention, the one or more lipids is or comprises one or more lipidated oligopeptides, particularly those having one or more amino acid residues that is or are cationic at a pH of 7.4 (physiological pH; examples include lysine and arginine).

The lipidated oligopeptide may be used in combination with one or more phospholipids, such as DOPE or DPPC. It is thought that the alkyl chain of a lipopeptide may advantageously be assimilated in a phospholipid bilayer, while the surface of the bilayer is decorated with the peptide moiety. It is thought that in this way, the peptide may provide for tissue and/or cell targeting; and, for example where the peptide bears a cationic charge at physiological pH, may stabilise negatively charged APIs, such as nucleic acid, such as mRNA.

According to all aspects of the invention, there is or are present in the pharmaceutical composition one or more lipids. Preferably said one or more lipids is or are provided in association with the hydrolysable doped silicon particles of the invention.

According to certain embodiments the lipid is or comprises at least one cationic lipid; a helper lipid, e.g. a phospholipid; a structural lipid, e.g. a cholesterol-based lipid; and/or a polyethylene glycol (PEG) lipid.

The lipids may include one or more of: phosphatidylcholine (PC), hydrogenated PC, stearylamine (SA), dioleoylphosphatidylethanolamine (DOPE), cholesteryl 3β-N-(dimethylaminoethyl)carbamate hydrochloride (DC)-cholesterol, 1,2-dioleoyl-3-trimethylammonium-propane (DOTAP) and derivatives thereof. In certain embodiments, the lipid comprises or consists of DOTAP. It has been found that surface treating the particle with a lipid aids in controlling the rate of release of the nucleic acid or other pharmaceutically active agent. The type of lipid used to treat the surface of the silicon containing particle may affect its rate of release. In particular, surface treating a hydrolysable doped silicon particle with a lipid has a beneficial effect on the surface charge of the particles, providing them with the requisite zeta potential to allow for improved loading of short interfering RNA or short activating RNA or short hairpin RNA or messenger RNA, and controlling the rate of their release at a target site. The presence of at least one lipid may also allow for the rate of hydrolysis of the doped silicon to be controlled, such that the doped silicon hydrolyses to the bioavailable orthosilicic acid (OSA) degradation product rather than insoluble polymeric hydrolysis products. Controlling the rate of hydrolysis of the doped silicon will influence the rate at which nucleic acid or other active pharmaceutical ingredients associated with the doped silicon is released. Controlling the rate of release will affect how long the protection of the active pharmaceutical ingredient is sustained for.

Nevertheless, doped silicon as used herein provides the ability to use less lipid (in particular lower levels of cationic lipids, such as DOTAP), than conventional compositions comprising active pharmaceutical ingredients yet without hydrolysable doped silicon particles, particularly conventional transfection compositions comprising nucleic acid; and/or provides the ability for such compositions to be formulated with a wider range of lipids while still providing transfection efficiency, storage stability, and/or targeted delivery to a particular type of tissue, or to a particular type of cell. Thus there is provided a way to reduce reliance on specific lipids, particularly cationic lipids, such as DOTAP. Thus, in certain embodiments of all aspects of the invention, the one or more lipids are selected from the group consisting of: a helper lipid, e.g. a phospholipid; a structural lipid, e.g. a cholesterol-based lipid; and/or a polyethylene glycol (PEG) lipid. In such embodiments, optionally, none of the one or more lipids are cationic lipids such as DOTAP.

In certain embodiments of all aspects of the invention, the lipid is DOTAP or the lipids present in the formulation comprise DOTAP. DOTAP exists in an S and an R enantiomeric form, and may be present according to all aspects of the invention as the S-, R-form or as a racemate. According to certain embodiments of the total DOTAP present in a composition of the invention the R and S forms may be in approximately equal amounts (i.e. no more than 60% of either form). In other embodiments at least 80, 90, 95, 98, or 99% of total DOTAP is in the R-form. In other embodiments at least 80, 90, 95, 98, or 99% of total DOTAP is in the S-form. Where the one or more lipids is, are or comprise a cationic lipid, such as DOTAP, doping of the silicon particles in accordance with all aspects of the present invention preferably enables less of the cationic lipid, such as DOTAP, to be used than in pharmaceutical compositions for delivery of an active pharmaceutical ingredient, such as a nucleic acid, which do not comprise hydrolysable doped silicon particles.

According to all aspects of the invention, the lipid or lipids may have an average molecular weight in the range of 500 to 1000.

Preferably, and in accordance with all aspects of the invention (for example, when the lipid contains one or more of a cationic lipid, a helper lipid, a structural lipid and a PEG lipid, or is selected from one or more of PC, hydrogenated PC, SA, DOPE, DOTAP, DC-cholesterol, and derivatives thereof) the ratio of lipid (i.e. total lipid components) to doped before any further processing (i.e. by means of filtration or sterilization process) is between 1:1 and 45:1, for example between 1:1 and 20:1, 1:1 and 16:1, 1:1 and 12:1, 1:1 and 11:1, 1:1 and 10:1, 1:1 and 9:1, 1:1 and 8:1, 1:1 and 13:1, 2:1 and 12:1, 2:1 and 11:1, 2:1 and 10:1, 2:1 and 9:1, 2:1 and 8:1, for example between 1:1 and 7:1, between 2:1 and 7:1, between 3:1 and 6:1, between 4:1 and 5:1. It has been found that lipid component to silicon molar ratios of between 0.8:1 and 20:1 are particularly advantageous, for example 16:1, 12:1, 8:1, or 2.5:1.

Advantageously, this ratio of lipid to doped silicon may provide a multilamellar vesicle system able to control the release of, and stabilise, the active pharmaceutical ingredient (for example nucleic acid) in contact with the particle of hydrolysable doped silicon and to facilitate the controlled the release of the bioavailable degradation product of the silicon, OSA.

Advantageously, the lipid compound can exert a significant effect on the surface charge of the doped silicon nanoparticles. Particles comprising hydrolysable pure silicon treated with phosphatidylcholine (PC), phosphatidylethanolamine (PE) and lecithin demonstrate a negative surface charge when zeta potential analysis is performed (ranging from −60 to −20 mV, with various preferred ratios of silicon:lipid). Particle surfaces treated with stearylamine or DOTAP demonstrate a positive zeta potential (ranging from 0 to +40 mV, with various preferred ratios of silicon:lipid). Doping of silicon changes the surface charge. Use of a p-dopant, such as boron (which is preferred in many embodiments of the invention in all of its various aspects) will make the zeta potential more positive (i.e. less negative). A typical value of −40 mV for pure silicon will become about −25 mV when the silicon is doped with boron. Therefore, boron doped silicon can more easily achieve a positive zeta potential when treated with a cationic lipid. For example, treatment with stearylamine or DOTAP can achieve a value of about +20 mV to +60 mV. This means that a positive surface zeta potential may be achieved with a lower amount of cationic lipid or with a wider range of cationic lipids (including those which are less cationic than stearylamine and DOTAP). It also means that even if the cationic lipid degrades (“ages”) during storage resulting in a partial loss of positive charge of the lipid, the surface zeta potential of the particle will remain more comfortably positive for longer.

It has been found that lipid component to silicon molar ratios of between 0.8:1 and 20:1 are particularly advantageous, for example 16:1, 12:1, 8:1, or 2.5:1.

The lipid or lipids component may, in some embodiments, be or comprise a phospholipid. The term “phospholipid” refers to a lipid comprising a fatty acid chain and a phosphate group. Phospholipids are typically neutral molecules in that they do not have an overall charge or they may carry a negative charge, unlike a cationic lipid which is positively charged. Phospholipids are typically zwitterionic compounds comprising both positive and negatively charged components, but no overall charge. As such, phospholipids are typically classified as neutral lipids. Particularly suitable phospholipids are glycerophospholipids. Particularly suitable phospholipids are those in which the polar head group is linked to quaternary ammonium moieties, such as phosphatidylcholine (PC) or hydrogenated phosphatidylcholine. Another example of a phospholipid is DOPE (phosphatidyl ethanolamine or 1,2-dioleoyl-sn-glycero-3-phosphoethanolamine). The type of lipid may be selected depending on the nature of the formulation, with neutral or negatively charged phospholipids being preferred for aprotic formulations, while positively charged cationic lipids and small CH₃ chain lipids are preferred for protic formulations. The phospholipid may be, or be derived from lecithin.

Preferably, the side chain(s) of the phospholipid is/are (an) aliphatic side chain(s) with 15 or more carbon atoms, or an ether side chain with 6 or more repeating ether units, such as a polyethylene glycol or polypropylene glycol chain. Lipids with ether side chains may be referred to as “PEG-lipids” or “PEG-ylated” lipids. Thus, as used in the present application, the term “lipid” may thus cover PEG lipids. Thus, according to certain embodiments, the lipid is or comprises one or more polyethylene glycol (PEG) lipids.

The lipid or lipids component may, in some embodiments, be or comprise a cationic lipid. The term “cationic lipid” refers to positively charged molecules having a cationic head group attached via some spacer to a hydrophobic tail. Examples include DTDTMA (ditetradecyl trimethyl ammonium), DOTMA (2,3-dioleyloxypropyl-1-trimentyl ammonium), DHDTMA (dihexadecyl trimethyl ammonium) and stearylamine (SA). The positive charge is typically stabilised by a negative counter ion. In preferred embodiments the cationic lipid is, or comprises DOTAP. As described herein, doping of silicon in accordance with all aspects of the present invention may provide the ability to use lower levels of cationic lipid such as DOTAP, compared to conventional compositions, such as transfection compositions, which are formulated without hydrolysable doped silicon; yet the compositions comprising hydrolysable doped silicon as provided herein maintain adequate transfection ability (for example, good tissue targeting ability or good cell targeting ability) and/or good storage stability.

In certain embodiments, the lipid is selected from the group consisting of phosphatidylethanolamine (PE), phosphatidylcholine (PC), stearylamine (SA), or any combination thereof.

In certain embodiments the lipid may consist substantially of phosphatidylcholine, hydrogenated phosphatidylcholine, stearylamine, or combinations thereof.

In certain embodiments the lipid may consist of at least 5% by weight of hydrogenated phosphatidylcholine, for example at least 20 wt %, typically at least 30 wt % and especially at least 50 wt % hydrogenated phosphatidylcholine based on the total weight of the particle. It has been found that hydrogenated phosphatidylcholine to doped silicon molar ratios of between 0.8:1 to 5:1 are particularly advantageous, for example 1:1, 1.5:1, 2:1, 2.5:1, 3:1, 3.5:1, 4:1, or 4.5:1.

In certain embodiments the lipid may consist of at least 5% by weight phosphatidylcholine, for example at least 20 wt %, typically at least 30 wt % and especially at least 50 wt % phosphatidylcholine, based on the total weight of the particle. It has been found that phosphatidylcholine to doped silicon molar ratios of between 0.8:1 to 5:1 are particularly advantageous, for example 1:1, 1.5:1, 2:1, 2.5:1, 3:1, 3.5:1, 4:1, or 4.5:1.

In certain embodiments the lipid may consist of at least 5% by weight of stearylamine, for example at least 20 wt %, typically at least 30 wt % and especially at least 50 wt % stearylamine based on the total weight of the particle. It has been found that stearylamine to doped silicon molar ratios of between 0.8:1 to 5:1 are particularly advantageous, for example 1:1, 1.5:1, 2:1, 2.5:1, 3:1, 3.5:1, 4:1, or 4.5:1. In certain embodiments the lipid may consist of PC and SA, preferably in a ratio by weight of PC: SA of from 1:1 to 20:1, more preferably 7:1 to 10:1, such as a ratio by weight of PC: SA of 72:8.

In certain embodiments the lipid may consist of DOPE, SA, and DC-cholesterol. The ratio by weight of DOPE: SA may be in a range of from 1:1 to 10:1, for example from 4:1 to 8:1. The ratio by weight of DOPE: DC-cholesterol may be in a range of from 1:1 to 5:1, for example from 1:1 to 3:1. The ratio by weight of SA: DC-cholesterol may be in a range of from 1:1 to 1:5, for example from 1:2 to 1:4. In some embodiments, the ratio by weight of DOPE: SA: DC-cholesterol may be 48:8:24.

In certain preferred embodiments the lipid may consist of DOTAP, DOPE and a PEG-lipid (such as mPEG2000-DSPE). The weight ratio of DOTAP: DOPE may be from 1:2 to 2:1, for example approximately 1:1. The ratio of DOTAP: PEG-lipid and DOPE: PEG-lipid may be 10:1 to 5:1, for example approximately 7:1. The total weight ratio of total lipid to silicon may be between 20:1 and 10:1, for example approximately 16:1.

### Amino Acids

All aspects of the invention may include the additional optional presence or use of one or more amino acids.

In its broadest sense, the term “amino acid” encompasses any artificial or naturally occurring organic compound containing an amine (—NH2) and carboxyl (—COOH) functional group. It includes α, β, γ and δ amino acids. It includes an amino acid in any chiral configuration. According to some embodiments (for example, when the doped silicon particles of the invention are formulated with one or more of PC, hydrogenated PC, SA, DOPE, DC-cholesterol, and derivatives thereof) the amino acid is preferably a naturally occurring a amino acid. It may be a proteinogenic amino acid or a non-proteinogenic amino acid (such as carnitine, levothyroxine, hydroxyproline, ornithine or citrulline). In preferred embodiments the amino acid comprises arginine, histidine, or glycine, or a mixture of arginine and glycine. In particularly preferred embodiments, the amino acid comprises glycine. Such amino acids may function to stabilise the doped silicon particle and control the hydrolysis of doped silicon both in storage and in vivo.

In addition to the amino acids described above, all aspects may include a peptide containing a cell surface receptor-for example integrin-recognition sequence that confers a degree of cell specificity to the particle. The peptide may have a “head group” containing a cell surface receptor recognition sequence and additionally a “tail” that can bind non-covalently to the active pharmaceutic agent, for example a nucleic acid and/or which binds to the doped silicon.

### Active Pharmaceutical Agent—Particle Association

According to preferred embodiments (for example, where particles comprising hydrolysable boron-doped silicon are formulated with one or more amino acids, e.g. arginine, glycine and histidine, and/or one or more lipids, e.g. PC, hydrogenated PC, SA, DOPE, DOTAP, DC-cholesterol, and derivatives thereof, or other ionisable or cationic lipids) at least 70%, for example at least 80%, for example at least 90% of the active pharmaceutical agent (for example, the nucleic acid, saRNA, shRNA, siRNA or mRNA) by weight present in the products of all aspects of the invention is associated with the particles.

By this, it is meant that the active pharmaceutical agent is non-covalently associated with the doped silicon. Without wishing to be bound by theory, it is hypothesised that when this takes place, the random Brownian movement of the active pharmaceutical agent, such as nucleic acid, may be reduced and the efficiency of the pharmaceutical composition is enhanced. For example, opportunities for it to be degraded, for example by degradative enzymes present in a formulation, are reduced. Also without wishing to be bound by theory, it is thought that there may be a reduction in the number of water molecules available for enzyme-catalysed reactions with the active pharmaceutical agent (for example, nucleic acid and particularly mRNA). For example, water molecules may be removed by reaction of water with hydrolysable doped silicon.

The rate of degradation of the particle and the end of its association with the active pharmaceutical agent which results from the degradation is governed by the hydrolysis of the doped silicon in the particles. As this rate can be controlled, the rate at which the active pharmaceutical agent becomes bio-available can also be controlled in order to avoid dose-dumping and/or to ensure gradual release over a suitably long period of time.

Where the active pharmaceutical agent is a nucleic acid, treating the lipid-treated boron-doped silicon particles (for example, nanoparticles treated with one or more of PC, hydrogenated PC, SA, DOPE, DOTAP, DC-cholesterol, and derivatives thereof) with an amino acid (for example, one or more of glycine, arginine and histidine, preferably glycine) has been found to provide a beneficial stabilising effect on a nucleic acid such as RNA (for example, mRNA saRNA, shRNA or siRNA). In particular, treating the lipid-treated particles with amino acids has been shown to stabilise nucleic acid such as RNA in biological fluids, for example in ocular tissues or blood plasma. Lipid-treated particles formulated with an amino acid in this manner may be particularly suitable for delivery to the body, for example delivery by transcutaneous injection.

### Ratio of Amino Acid(s) to Doped Silicon

Preferably (for example, when the particles of the invention are formulated with one or more amino acids, e.g. one or more of arginine, histidine, and glycine; and/or one or more lipids, e.g. one or more of PC, hydrogenated PC, SA, DOTAP, DOPE, DC-cholesterol, and derivatives thereof) the amino acid (for example glycine, or a mixture of glycine and lysine) is optionally present at a weight ratio to silicon of at least 500:1, at least 50:1, at least 5:1, at least 2.5:1, at least 1:1, or at least 0.5:1 or 0.05:1. Preferably, the amino acid is glycine which is optionally present a weight ratio to silicon of at least 500:1, at least 50:1, at least 5:1, at least 2.5:1, at least 1:1, or at least 0.5:1 or 0.05:1.

Advantageously, this ratio of amino acid to doped silicon further affects the rate of release of, and stabilises, an active pharmaceutical agent such as a RNA molecule associated with the particle.

According to all aspects of the invention, the particles may be treated with a lipid (for example, one or more of PC, hydrogenated PC, SA, DOTAP, DOPE, DC-cholesterol, and derivatives thereof) and an amino acid (for example, one or more of glycine, arginine and histidine, such as glycine or a mixture of glycine and arginine). The amino acid may be any amino acid. Preferably, the amino acid is arginine or glycine, or a combination of glycine and arginine. The lipid can be any lipid. Preferably, the lipid is a phospholipid. Optionally, the lipid further comprises a cationic lipid. More preferably, the lipid or lipids are selected from one or more of hydrogenated PC, PC, DOTAP, DOPE, lecithin, stearylamine, and derivatives thereof. Optionally, the lipid comprises DOTAP and/or a derivative thereof.

Preferably, the ratio of amino acid to doped silicon is between 0.05:1 to 0.4:1, for example between 0.08:1 and 0.35:1, especially 0.09:1 to 0.32:1. In some embodiments (for example, when the lipid is selected from one or more of PC, hydrogenated PC, SA, DOTAP, DOPE, DC-cholesterol, and derivatives thereof) the amino acid is a combination of arginine and glycine, wherein the ratio of Arg: Gly is between 1:0.6 and 3:1, for example between 1:0.8 and 2.5:1, for example between 1:1 and 2:1.

According to other embodiments of all aspects of the invention the particles are formulated with arginine. Preferably, the ratio of arginine to boron-doped silicon is between 0.05:1 to 0.4:1, for example between 0.08:1 and 0.35:1, especially 0.09:1 to 0.32:1.

According to other embodiments of all aspects of the invention the particles are formulated with glycine. Preferably, the ratio of glycine to boron-doped silicon is between 0.05:1 to 0.5:1, for example between 0.08:1 and 0.45:1, especially 0.09:1 to 0.42:1.

Preferred amino acids for use with all aspects of the invention include arginine, glycine and histidine and mixtures of two or more thereof.

### Active Pharmaceutical Agent

According to all aspects of the invention the active pharmaceutical agent (also referred to as the active pharmaceutical ingredient) may be any pharmaceutically active compound. In some embodiments it may be a pro-drug. In preferred embodiments of all aspects of the invention the active pharmaceutical agent is a nucleic acid.

Nucleic acids such as RNAs for use in accordance with the invention include double-and single-stranded DNA, RNA, DNA: RNA hybrids, and hybrids between PNAs (peptide nucleic acids) or RNA or DNA. The terms also include known types of modifications, for example, labels which are known in the art, methylation, “caps,” substitution of one or more of the naturally occurring nucleotides with an analogue, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides will also include modifications on the sugar moiety, e.g., wherein one or more of the hydroxyl groups are replaced with a halogen, an aliphatic group, or are functionalized as ethers, amines, or the like. Other modifications to nucleotides or polynucleotides involve rearranging, appending, substituting for, or otherwise altering functional groups on the purine or pyrimidine base which form hydrogen bonds to a respective complementary pyrimidine or purine, e.g., isoguanine, isocysteine, and the like. In some embodiments, the oligonucleotides and/or probes include at least one, two, three or four modified nucleotides.

In some embodiments, the nucleic acids such as the RNAs disclosed herein include one or more universal bases. As used herein, the term “universal base” refers to a nucleotide analog that can hybridize to more than one nucleotide selected from A, U/T, C, and G. In some embodiments, the universal base can be selected from the group consisting of deoxyinosine, 3-ntiropyrrole, 4-nitroindole, 6-nitroindole, 5-nitroindole.

According to preferred embodiments of all aspects of the invention the nucleic acid may be DNA or RNA. In preferred embodiments, the nucleic acid is RNA. RNA includes, in various embodiments of all aspects of the invention, mRNA, saRNA, shRNA and siRNA. Preferably, the nucleic acid is mRNA. In preferred embodiments, it may be the mRNA of an mRNA vaccine.

In its broadest sense, the term saRNA encompasses small activating RNA, comprising double-stranded RNA molecules of 5 to 50 base pairs in length, which operates within the RNA activation (RNAa) pathway. For example, the saRNA may be 10 to 45 base pairs, 15 to 40 base pairs, or 20-30 base pairs, especially 20 to 25 base pairs in length.

In its broadest sense, the term shRNA encompasses single stranded RNA molecules having base pairing and haing a length of 10 to 100 bases, which operate within the RNA interference (RNAi) pathway. For example, the shRNA may be 15 to 95 base pairs, 20 to 80 base pairs, or 25 to 75 base pairs, especially 30 to 70 base pairs in length.

In its broadest sense, the term “siRNA” encompasses small interfering RNA (siRNA), sometimes known as short interfering RNA or silencing RNA: comprising double-stranded RNA molecules of 5 to 50 base pairs in length: and operates within the RNA interference (RNAi) pathway. For example, the siRNA may be 10 to 45 base pairs, 15 to 40 base pairs, or 20-30 base pairs, especially 20 to 25 base pairs in length.

The term “mRNA” encompasses messenger RNA and may optionally include mRNA comprising a 5-prime cap and/or a poly-adenylated terminus. Alternatively, one or both of those features may be absent, mRNA may be in certain embodiments at least 100, at least 200, at least 300, at least 500, or at least 1000 base pairs in length.

RNA according to preferred embodiments of all aspects of the invention (for example, when the particles of the invention are formulated with one or more of arginine, histidine, and glycine, and/or one or more of PC, hydrogenated PC, SA, DOTAP, DOPE, DC-cholesterol, and derivatives thereof) may be naturally occurring or chemically modified to enhance their therapeutic properties, such as enhanced activity, increased serum stability, reduced off-targeting and lower immunological activation. Chemical modifications to the RNA may include any modifications commonly known in the art.

According to certain embodiments (for example, when the particles of the invention are formulated with one or more of arginine, histidine, and glycine, and/or one or more of PC, hydrogenated PC, SA, DOPE, DOTAP, DC-cholesterol, and derivatives thereof) the RNA is siRNA. According to other embodiments (for example, when the particles of the invention are formulated with one or more of arginine, histidine, and glycine, and/or one or more of PC, hydrogenated PC, SA, DOTAP, DOPE, DC-cholesterol, and derivatives thereof) the RNA is mRNA.

According to other embodiments the mRNA encodes an antigen, thereby providing a pharmaceutical composition which is a vaccine. The antigen may be a viral antigen, for example an antigen of SARS-CoV-2, for example an antigen deriving from the spike protein of SARS-CoV-2.

In certain embodiments the mRNA may encode multiple proteins. For example, the mRNA may encode a viral antigen and an adjuvanting protein, or multiple viral antigens. In other embodiments an adjuvant may alternatively or additionally be provided as a additional component of the pharmaceutical composition in addition to the active pharmaceutical agent.

### Ratio of Doped Silicon to Active Pharmaceutical Agent

Preferably (for example, when the particles of the invention are formulated with one or more of arginine, histidine, and glycine, and/or one or more of PC, hydrogenated PC, SA, DOTAP, DOPE, DC-cholesterol, and derivatives thereof) the ratio of doped (for example boron-doped) silicon to the active pharmaceutical agent such as nucleic acid (such as siRNA or mRNA) is between 1:1 and 8:1, for example between 1:1 and 6:1, 1:1 and 5:1, 1:1 and 4:1, or between 1:1 and 3:1. Preferably, the ratio of doped (for example boron-doped) silicon to active pharmaceutical agent such as nucleic acid is between 1:1 and 3:1. Advantageously, this ratio of doped (for example boron-doped) silicon to pharmaceutical agent such as nucleic acid further affects the rate of release of, and stabilises, the active pharmaceutical agent, for example nucleic acid molecule, (such as an siRNA, saRNA, shRNA or mRNA molecule) conveyed by the particle.

It should be noted that in all of these ratios (and in other ratios throughout this specification) the ratios are weight ratios and the ratio ascribed to “silicon” is the total weight of the hydrolysable doped silicon containing particle and is measured before any additional preparative steps such as sterilization or filtration process which may alter the subsequent ratios.

### Preferred Combinations

According to all aspects of the invention, especially preferred embodiments relate to doping which is boron doping (especially heavy boron doping as defined above), and an active ingredient which is a nucleic acid (especially mRNA, and especially a mRNA encoding an antigen of a mRNA vaccine). Optionally, the lipid is or comprises DOTAP. However, it has been found that DOTAP is not always necessary for the present formulations, as shown in the Examples hereinbelow, particularly Example 6. In accordance with the Examples, therefore, in especially preferred embodiments, the one or more lipids is, are or comprise(s) one or more of a phospholipid (such as DPPC and/or DOPE) and a lipidated oligopeptide having one or more amino acid residues that is or are cationic at a pH of 7.4 (physiological pH: examples include lysine and arginine). Optionally also present are one or more sugars (particularly trehalose) and/or one or more amino acids (particularly glycine).

Alternatively, in other preferred embodiments, the one or more lipids are or comprise one or more of a phospholipid (such as DPPC and/or DOPE), and are formulated with one or more coenzymes (for example, NAD): one or more flavanols (for example, quercetin) and/or one or more amino acids (for example, tyrosine). Optionally also present are one or more sugars (particularly trehalose) and/or one or more amino acids (particularly glycine).

### Enhancement of the Efficiency of a Pharmaceutical Composition

In some aspects, the invention relates to the realisation that a particle comprising hydrolysable doped (for example boron doped) silicon can be effective in enhancing the efficiency of a pharmaceutical composition comprising an active pharmaceutical ingredient.

The invention thus provides in a seventh aspect the use of particles comprising hydrolysable doped silicon, optionally hydrolysable boron doped silicon, to enhance the efficiency of a pharmaceutical composition comprising an active pharmaceutical ingredient and likewise a method of enhancing the efficiency of a pharmaceutical composition comprising an active pharmaceutical ingredient by incorporating particles comprising hydrolysable doped silicon in the pharmaceutical composition. The efficiency of the pharmaceutical composition may be enhanced by means of the particles increasing the stability at room temperature (or 4° C.) of the active pharmaceutical ingredient and/or enhanced by means of the particles enhancing the uptake of the active pharmaceutical ingredient by a target cell or tissue, since the particles comprising hydrolysable doped silicon enhance tissue or cell targeting ability. The efficiency of the pharmaceutical composition may also or alternatively be enhanced by means of the particles increasing the intracellular stability of the active pharmaceutical ingredient and/or by means of the particles protecting the active pharmaceutical ingredient from degradation, for example enzymatic degradation. The pharmaceutical composition is optionally as defined herein with reference to all aspects of the invention. In this way, optionally, there may be provided the ability to use lower levels of cationic lipid, such as DOTMA or DOTAP, compared to conventional transfection compositions, such as transfection compositions, which do not comprise hydrolysable doped silicon, whilst still maintaining adequate transfection ability (for example, good tissue targeting ability or good cell targeting ability) and/or good storage stability.

### Further Components and Features

According to preferred embodiments of all aspects of the invention, wherein the active pharmaceutical agent is a nucleic acid there may additionally be present one or more further components, including additional transfection reagents.

In its broadest sense, “transfection reagents” are agents that facilitate the introduction of naked or purified nucleic acids into eukaryotic cells. For example, some transfection reagents are agents that facilitate the induction of mRNA into eukaryotic cells.

According to other embodiments of all aspects of the invention (for example, when boron-doped silicon nanoparticles of the invention are formulated with one or more of arginine, histidine, and glycine, and/or one or more of PC, hydrogenated PC, SA, DOTAP, DOPE, DC-cholesterol, and derivatives thereof) the transfection reagents may be lipofection (liposome transfection) reagents, dendrimers, a HEPES-buffered saline solution (HeBS) containing phosphate ions combined with a calcium chloride solution, or cationic polymers such as diethylaminoethyl-dextran (DEAE dextran) or polyethylenimine (PEI).

In preferred embodiments (for example, when boron-doped silicon particles of the invention are formulated with one or more of arginine, histidine, and glycine, and/or one or more of PC, hydrogenated PC, SA, DOTAP, DOPE, DC-cholesterol, and derivatives thereof) the transfection reagent is a lipofection reagent, such as lipofectamine.

The nucleic acids according to preferred embodiments of all aspects of the invention, such as the RNAs (such as siRNA, saRNA, shRNA or mRNA) for use in various aspects of the invention may be provided in various forms. For example, in some embodiments (for example, when the particles of the invention are formulated with one or more of arginine, histidine, and glycine, and/or one or more of PC, hydrogenated PC, SA, DOPE, DOTAP, DC-cholesterol, and derivatives thereof) the nucleic acids such as RNAs are provided in solution (either alone or in combination with various other nucleic acids), for example buffer. In some embodiments (for example, when boron-doped silicon nanoparticles of the invention are formulated with one or more of arginine, histidine, and glycine, and/or one or more of PC, hydrogenated PC, SA, DOTAP, DOPE, DC-cholesterol, and derivatives thereof) nucleic acids such as RNAs are provided, either alone or in combination with other isolated nucleic acids, as a salt. In some embodiments (for example, when boron-doped silicon nanoparticles of the invention are formulated with one or more of arginine, histidine, and glycine, and/or one or more of PC, hydrogenated PC, SA, DOTAP, DOPE, DC-cholesterol, and derivatives thereof) nucleic acids such as RNAs are provided in a lyophilized form that can be reconstituted. For example, in some embodiments, the nucleic acids such as RNAs can be provided in a lyophilized pellet alone, or in a lyophilized pellet with other isolated nucleic acids. In some embodiments (for example, when the particles of the invention are formulated with one or more of arginine, histidine, and glycine, and/or one or more of PC, hydrogenated PC, SA, DOTAP, DOPE, DC-cholesterol, and derivatives thereof) nucleic acids such as RNAs are provided affixed to a solid substance, such as a bead, a membrane, or the like. In some embodiments (for example, when the particles of the invention are formulated with one or more of arginine, histidine, and glycine, and/or one or more of PC, hydrogenated PC, SA, DOPE, DOTAP, DC-cholesterol, and derivatives thereof) nucleic acids such as RNAs are provided in a host cell, for example a host cell of a cell line carrying a plasmid, or a host cell of a cell line carrying a stably integrated sequence.

Pharmaceutical compositions of the invention may include further excipients, including but not limited to: preservatives, cryoprotectants and adjuvants.

Pharmaceutical compositions of the invention containing siRNA may optionally include siRNA which is synthetically manufactured by chemical synthesis outside of a biological system. Such siRNA can be manufactured free of nucleic acid degradative enzymes.

However, the preferred way of manufacturing longer nucleic acids such as mRNA (for example for use in vaccine formulations) involves the use of a biological system. The mRNA is typically purified from that biological system in order to reduce the level of degradative enzymes (for example RNases). It may be difficult to completely eliminate all degradative enzymes. This would normally necessitate storage at low temperature to minimise enzyme activity. Moreover, during translocation around the body and/or escape from the endosomal compartment, nucleic acid is exposed to physiological and intracellular conditions, typically including contact with degradative enzymes.

However, it has been found that formulating nucleic acid (such as mRNA) with particles comprising hydrolysable boron-doped silicon and ome or more lipids (and optionally a non-reducing disaccharide) according to the invention in its various aspects, can increase the storage life of the mRNA in a formulation, possibly negating, mitigating or reducing the need for low temperature storage; and/or stabilising the mRNA during translocation around the body and/or escape from the endosomal compartment.

Accordingly, methods of the invention include methods of protecting an active pharmaceutical ingredient which is a nucleic acid (for example mRNA) from degradation due to enzymes present in the nucleic acid preparation. Products of aspects of the invention may also be such that the nucleic acid is protected from degradation due to enzymes present in the nucleic acid-containing composition.

Preferably, enzymatic degradation at room temperature (20° C.) is reduced by at least half, more preferably by a factor of at least 5, 10, 35, 50, 100, 500 or 1000 compared to an equivalent composition without the particles of hydrolysable doped (e.g. boron doped) silicon-containing material. Preferably, the nucleic acid (such as mRNA) in the composition has a half-life at 4° C. of at least 3 months, at least 6 months or at least 12 months.

Certain embodiments of pharmaceutical compositions according to the first and other aspects of the invention contain nucleic acid (such as mRNA) as the active pharmaceutical agent from a biological source and a low but measurable level of degradative enzyme deriving from that biological source. For example, there may be one or more degradative enzymes present, which comprise one or more enzymes having an activity, on a nucleic acid substrate, of at least 1 μmol min⁻¹ at a pH of 7.4 and a temperature of 25° C.

### Proposed Mechanism of Action

Without wishing to be bound by theory, it is proposed that a process of “ensilication” may be part of the explanation of why boron doped silicon can prolong mRNA shelf life.

Ensilication consists of the formation of a protective and resistant silica “cage” around the mRNA, the silica cage being a product of partial hydrolysis of the doped silicon particle. Ensilication may confer protection against temperature-induced loss of structure and function without the need for freezing or refrigeration. In ensilication processes negatively charged silanol groups are able to participate in non-covalent interactions either directly with the pharmaceutically active agent or with lipid in the composition.

The resulting combination is able to physically prevent thermal denaturation of the nucleic acid or other agent.

To obtain further and specific improvements of the ensilication process, some characteristics of the silica material can be tailored to the compound to be protected. In particular, the use of boron-doped silicon allows the creation of a permanent cationic site able to form electrostatic interactions with nucleic acids thus protecting them from degradation.

The interaction of boron with a silicon cluster is responsible for a silica cage formation. Three boron atoms tend to form B₃ triangles encapsulated into Sin cages with increasing numbers of silicon atoms. Doping boron inside the silicon matrix results in a positive charge within the crystal structure, which allows binding of the nucleic acid within the crystal structure of silicon itself. This mitigates current problems caused by the degradation or “aging” of the lipid, thus increasing the lifespan of the nucleic acid-complexed delivery system.

### Preparation of Particles Comprising Doped Hydrolysable Silicon

The particles of the invention may conveniently be prepared by techniques conventional in the art, for example by milling processes or by other known techniques for particle size reduction. The doped silicon-containing particles may be made from sodium silicate particles, colloidal silica, magnesium reduction of silica or silicon wafer materials. Macro or micro scale particles maybe ground in a ball mill, a planetary ball mill, or other size reducing mechanism. The resulting particles may be sieved or air classified to recover particles. It is also possible to use plasma methods and laser ablation for the production of particles.

Porous particles may be prepared by methods conventional in the art, including the methods described herein.

An example specification of a boron-doped silicon for use according to the invention is:
single side polished Wafer, CZ
Diameter: 150±0,2 mm
Orientation: (100)+1°
Type: p/boron
resistivity: 0,014±25% Ohm cm. Close to 5×10¹⁸ Atoms/cm³.
Primary flat: 57,50±2,5 mm
Primary flat1 Location: D<100>to {110}
Thickness: 675±15 μm
packing: Ultrapak Shipping Casette
TTV: <=18 μm
TIR: <=5 μm

Such boron doped silicon is available commercially, for example from Nanografi, Jena, Germany or Si-Mat, Germany.

Various aspects and embodiments of the invention are now described with reference to the following non-limiting examples. Where the examples use non-doped silicon they may fall outside the scope of some aspects of the invention, but are included as comparative examples.

### EXAMPLES

### Example 1—Preparation of Boron Doped Silicon Particles

Single side boron-doped polished silicon wafers were purchased from Si-Mat, Germany. All cleaning and etching reagents were clean room grade. Etched silicon wafers were prepared by anodically etching Si in a 1:1 (v/v) mixture of pure ethanol and 10% aqueous HF acid for 2-10 min at an anodic current density of 80 mA/cm². After etching, the samples were rinsed with pure ethanol and dried under a stream of dry high-purity nitrogen prior to use. Etching can also be done in mixtures up to 1:3 solvent to 50% HF. Typical solvents are ethanol and isopropyl alcohol but other apriotic solvents like DMSO or cyclopentanone may also be used to achieve desired particle morphologies

Etched silicon wafers were crushed using a milling ball and/or a pestle & mortar. The fine powder was sieved using a Retsch™ sieve shaker AS 200 with gauge 38 μm. Uniform particle size selection (20-100 μm) is achieved by the aperture size of the sieve. The particle sizes were measured by the quantachrome system and PCS from Malvern Instruments. Samples were kept in a closed container until further use.

Nano silicon powder was also obtained from Sigma and Hefei Kaier, China. The particle size was measured by PCS and the size of the particles was recorded (size ranged between 20-100 nm) before being subjected to loading and etching.

500 mg of 100 nm diameter porous silicon nanoparticles were mixed with 250 ml ethanol and stirred using a magnet bar for 30 minutes. The solution was then centrifuged for 30 minutes at 3000 rpm. The supernatant was discarded and the nanoparticles were washed in 5 mL of distilled water and transferred to a round bottomed flask. The contents of the flask were frozen (2 hours at −25° C.). The frozen nanoparticles were freeze-dried using a freeze dryer overnight. The resultant dry powder is activated silicon nanoparticles.

### Example 2—Preparation of Boron Doped Silicon Particles

Wafers of boron doped silicon were obtained from BS Silicon prepared to the following specification:
single side polished wafer, CZ
Diameter: 150±0,2 mm
Orientation: (100)±1°
Type: p/boron
Resistivity: 0,014±25% Ohmcm. Close to 5×10¹⁸ boron atoms/cm³.
Primary flat: 57,50±2,5 mm
Primary flat1 Location: D <100>to {110}
Thickness: 675±15 μm
Packing: Ultrapak Shipping Casette
TTV: <=18 μm
TIR: <=5 μm

Undoped silicon was obtained from American Elements (Manchester, UK)

The silicon wafers were etched electrically (as in Example 1) to achieve at least 40% porosity and then crushed using a milling ball and/or a pestle & mortar. The fine powder was sieved using a Retsch™ sieve shaker AS 200 with gauge of 38 μm. Uniform particles sized from 20μm to 100μm) were selected by the sieve.

The powder was then activated by being treated with methanol (50 mg in 5 mL) and left under a fume hood thus allowing a slow evaporation process. The resulting powder was dispersed in nuclease free water at concentration of 1 mg/mL, in the presence of a non-reducing disaccharide (trehalose) and an amino acid (glycine).
DOTAP solution: DOTAP was solubilised in methanol at the concentration 5 mg/mL. In particular, 50 mg of DOTAP were solubilised in 10 ml of methanol and sonicated until fully solubilised.
DOPE solution: DOPE was solubilised in methanol at the concentration 5 mg/mL. In particular, 60 mg of DOPE were solubilised in 12 ml of methanol and sonicated until fully solubilised.
mPEG2000-DSPE solution: mPEG2000-DSPE was solubilised in methanol at the concentration 5 mg/ml. In particular, 40 mg of mPEG2000-DSPE were solubilised in 8 mL of methanol and sonicated until fully solubilised.

Trehalose was used as provided by Sigma Aldrich.

Glycine was used as provided by Sigma Aldrich.

Activated silicon particles were suspended with glycine and trehalose in nuclease-free water. In particular, 50 mg of activated silicon particles, 50 mg of trehalose and 25 mg of glycine were suspended in 50 mL of nuclease-free water and sonicated for 60 minutes.

siRNA specific for ClCn7G213R (see Capulli et al., 2015, Clin. Molec. Therap. 4, e248 for details) was then loaded onto the transfection vehicles

In vivo study—head to head transfection comparison using boron doped and undoped silicon particles as prepared in Example 2 in formulations for gene therapy.

Osteopetrosis Type 2 is modelled using ADO2 mutant mice which can be treated with siRNA specific for ClCn7G213R, if that siRNA is transfected into cells successfully. This model can therefore be used to assess transfection efficiency.

Transfection vehicles were prepared using boron-doped particles or control-non-doped particles in accordance with the invention, DOTAP, DOPE, mPEG2000-DSPE, glycine, trehalose and nuclease-free water. The vehicles were either administered empty or with an siRNA specific for ClCn7G213R.

Samples were coded as:
ADO2+SIS0012−Empty—undoped Si containing vehicle
ADO2+SIS0012−siRNA—undoped Si containing vehicle with siRNA specific for ClCn7G213R
ADO2+SIS0013−Empty—boron-doped Si containing vehicle
ADO2+SIS0013−siRNA—boron-doped Si containing vehicle with siRNA specific for ClCn7G213R 10 days old ADO2 mice were intraperitoneally injected with one of these constructs 3 times per week for 2 weeks (n=5 mice per group). psl Results

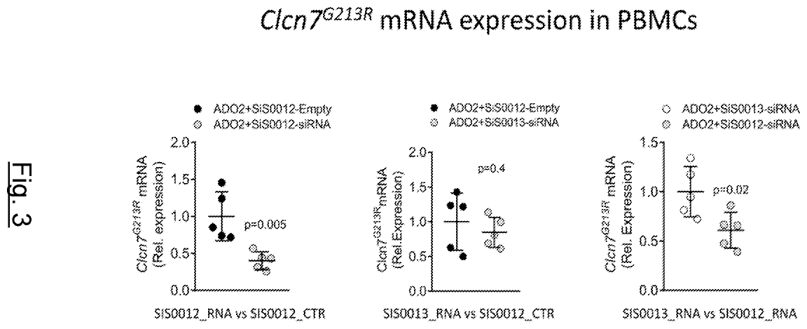

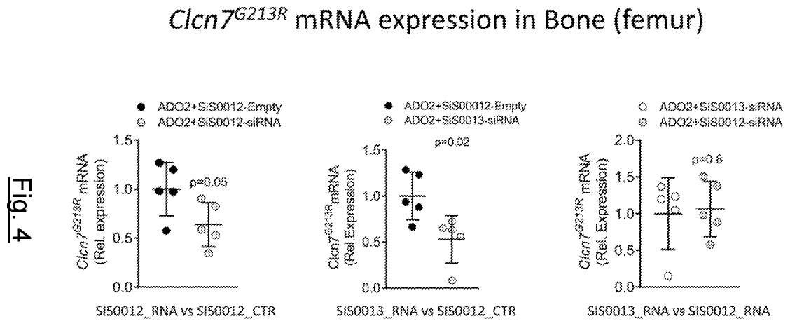

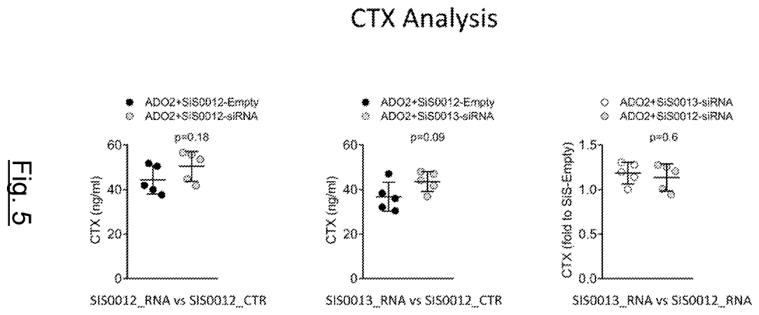

ClCn7G213R expression in mouse PMBCs was assayed for each treatment group and the results are presented in FIG. 3. FIG. 4 shows bone expression of ClCn7G213R and FIG. 5 shows CTX-blood test results (CTX is a marker for bone turnover)

ADO2SIS0012-siRNA complexes were able to significantly downregulate expression of relevant gene (ClCn7G213R) when compared to its relevant control group (ADO2SIS0012 empty, no SiRNA administered) in the target organ (femur, p<0.05). In addition, significant downregulation of the same gene in peripheral blood monocytes cells (PBMC, p<0.005 when ADO2SIS0012-siRNA complex was compared with ADO2SIS0012 empty, no SiRNA) was observed. For the period analysed and the proposed administration regimen (2 weeks treatment only), bone resorption was also slightly elevated and it is expected for longer time periods that change would be more substantial.

When the performance of the ADO2SIS0012-siRNA complex (made with undoped porous silicon particles) was compared with the ADO2SIS0013-siRNA complex (made with boron doped silicon particles) at the target organ ADO2SIS0013, both compositions provided an appreciable effect (FIG. 4, p<0.02). Use of boron-doped silicon resulted in a faster bone turnover, when compared to the performance of non-doped porous silicon (FIG. 5, CTX evaluation). It therefore can be said that the superior storage properties provided by using silicon which has been doped with boron do not detract from the transfection efficiency or therapeutic effect of siRNA formulations.

### Example 3—Preparation of Biological pDNA Containing Formulations

Formulation type 1 (refered to as a “Biocourier” formulation was prepared using silicon (porous, activated, Average particle size <100 nm, doped and un-doped as in Example 2) in accordance with the ingredients of table 2.

TABLE 2
Composition of Biocourier type 1

| Lipids | | | Activated | | | |
|---|---|---|---|---|---|---|
| | | mPEG2000- | SiNPs AE | | | Nuclease-free |
| DOTAP | DOPE | DSPE | (≤100 nm) | Glycine | Trehalose | water |
| 7.25 mg | 7.30 mg | 1.45 mg | 1 mg | 0.5 mg | 1 mg | Up to 10 mL |

| | | | | |
|---|---|---|---|---|
| 1.45 mL | 1.46 mL | 0.29 mL | 1 mL | Up to 10 mL |

### Lipid Film Preparation

Lipids, as listed in Table 2 above, were transferred into a clean glass round bottom flask and mixed in solvent which was evaporated using a rotary evaporator in a water bath at 40° C. and under vacuum.

### Rehydration of Lipid Film

The silicon particles, glycine and trehalose in solution were added to the lipid film and if necessary, the final volume was adjusted to 10 mL, using nuclease free water. The flask was covered with parafilm and agitated in a water bath (60° C.) for 5 minutes. 1 mL of aliquots were stored in RNA-free Eppendorf tubes in the refrigerator. Before use, the suspension was passed through a membrane filters of the pore sizes 0.4 μm and 0.1 μm, 10 times through 0.4 μm and then 10 times through 0.1 μm at 60° C.

Loading the Vehicle with pDNA

A frozen pDNA sample having a size of 6768bp was thawed from −40° C. to room temperature.

The vehicle and pDNA were placed together in a sterile Eppendorf tube and gently vortexed for 15 seconds, then left for 30 min at room temperature, then stored in a refrigerator until usage

### Example 4—Storage stability study with pDNA

### Aim

To evaluate the storage stability of different formulations produced as described in Example 2 using both doped and undoped silicon, by agarose gel electrophoresis.

### Formulations Used

“Biocourier SIS0012”—formation containing undoped silicon particles to be loaded with pDNA
“Biocourier SIS0013”—formation containing boron doped silicon particles to be loaded with pDNA
“Biocourier MV10010”—same as Biocourier SIS0012, but from a different batch made by a contract manufacturer.

### Method

A. Preparation of pDNA-Biocourier Complexes

The pDNA-Biocourier formulations were prepared by mixing the pDNA solution (2 mg/mL) with the Biocourier with a lipid/DNA ratio of 7.2. The final concentration of DNA in the mixture was 0.275 mg/mL. The mixtures were incubated at room temperature for 30 min to allow for complete complexation and then stored either at room temperature or at 4° C. The samples were prepared in 20 μL aliquots for each measurement time point and stored separately to minimize the risk of cross-contamination upon storage and analysis.

### B. Agarose Gel Electrophoresis

The pDNA-Biocourier complexes were analyzed by agarose gel electrophoresis at designated time points (0 h, 6 h, 24 h, 48 h, 72 h, and 8 days). The naked pDNA stored in similar conditions was used as a control in all cases. The samples were loaded onto the E-Gel™ agarose gel (1%) in an E-Gel™ Power Snap Electrophoresis Device. The amount of each sample and the total content of pDNA loaded onto the gel at different time points are provided in the table below. The total amount of naked pDNA loaded onto the gel at each time point was also the same as the pDNA-Biocourier complexes. The gel was transilluminated and imaged at 3 min and 7 min using an E-Gel™ Power Snap electrophoresis camera.

Table showing the amounts of pDNA-Biocourier complexes loaded on the agarose gel and total amount of pDNA per well at different measurement time points.

| | Amount of | Amount of nuclease | Amount of DNA |
|---|---|---|---|
| Time (h) | sample (μL) | free water (μL) | (μg/well) |
| 0 | 20 | 0 | 5.5 |
| 6 | 20 | 0 | 5.5 |
| 24 | 20 | 0 | 5.5 |
| 48 | 10 | 10 | 2.25 |
| 72 | 10 | 10 | 2.25 |
| 192 | 10 | 10 | 2.25 |

### Results

Test 1. Stability of the pDNA-Biocourier Complexes at Room Temperature

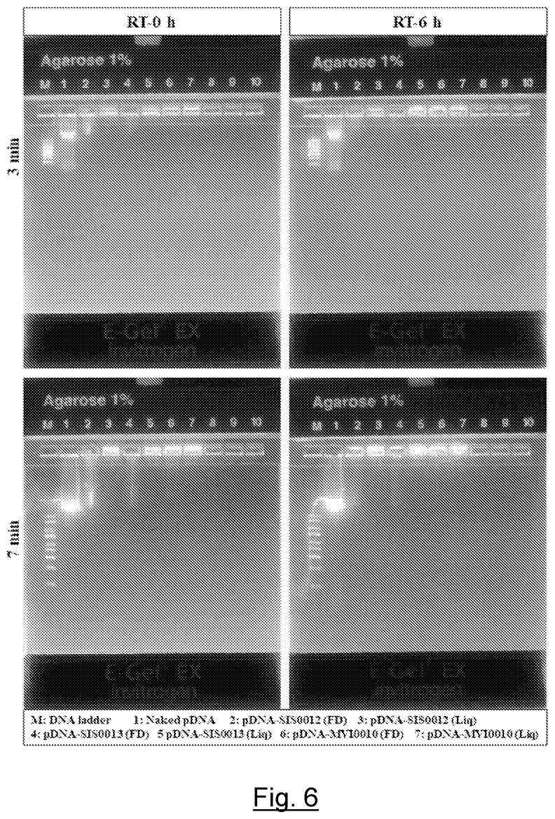

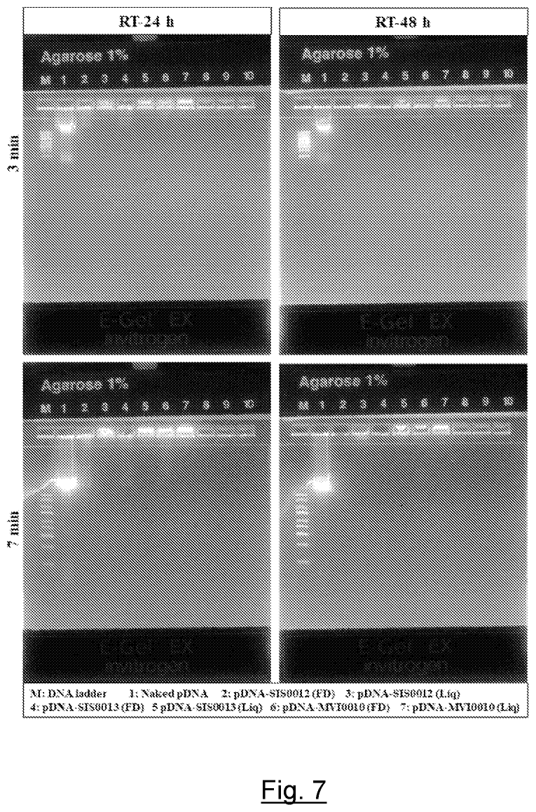

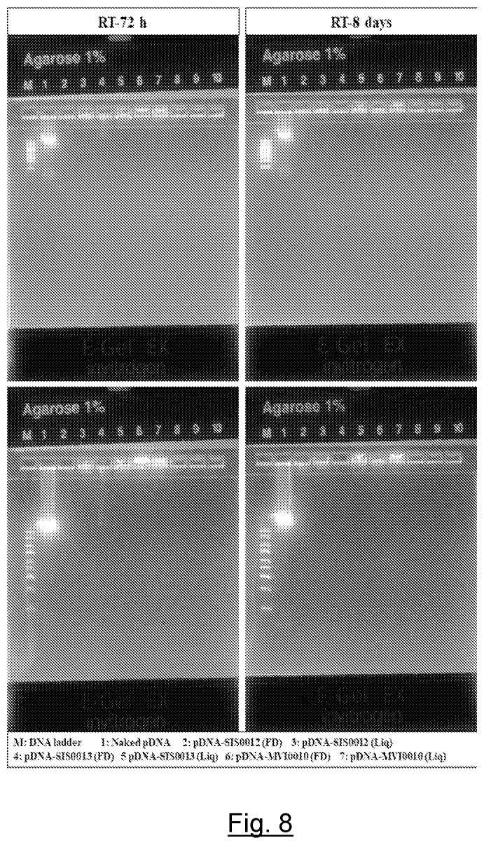

The agarose gel electrophoresis images of the pDNA-Biocourier formulations shortly after preparation and following storage at room temperature for 8 days are presented in FIGS. 6 to 8. All three Biocourier formulations in their liquid form completely retained the pDNA in the gel wells indicating full complexation. However, in the freeze-dried form, a small amount of pDNA passed through the gel indicating the presence of some unbound DNA. The higher intensity of the bands for SIS0012 and SIS0013 compared to MVI0010 indicates higher amount of unbound DNA. Nevertheless, comparing the intensity of the bands for the Biocourier formulations to the band from the naked pDNA which contains the same total amount of DNA as that loaded onto the Biocouriers indicates that a very small proportion of the total DNA loaded onto the Biocouriers is unbound which demonstrates a high binding efficiency. After 6 h storage at room temperature, the amount of unbound pDNA released into the gel from the freeze-dried formulations was less and could be due to degradation of pDNA at room temperature. The migration of unbound pDNA through the gel with the freeze-dried formulations continued until 24 h. However, from 48 h onwards no significant signal was observed for pDNA indicating a lack of DNA migration through the gel. It can be inferred from these data that although the freeze-dried formulations do not complex with the loaded pDNA completely, their binding efficiency was still very high as demonstrated by the negligible amount of DNA passing through the gel compared to the high amount of DNA loaded on the Biocouriers. Also, the pDNA-Biocourier complexes were stable at room temperature for 8 days as no more pDNA was released into the gel during that time which would have indicated dissociation.

Test 2. Stability of the pDNA-Biocourier Complexes at 4° C.

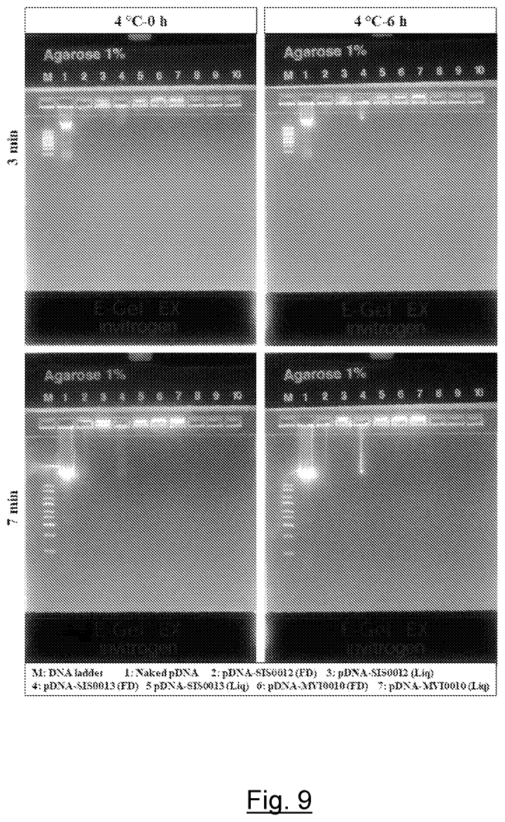

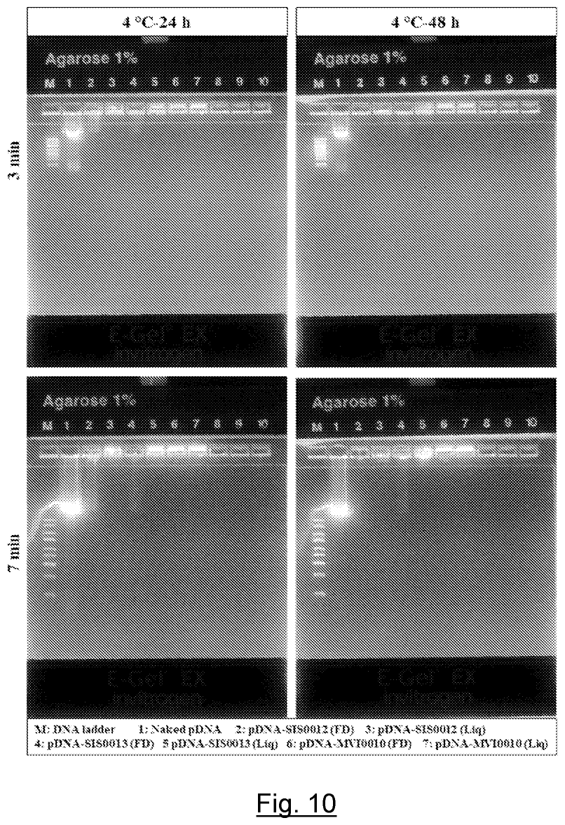

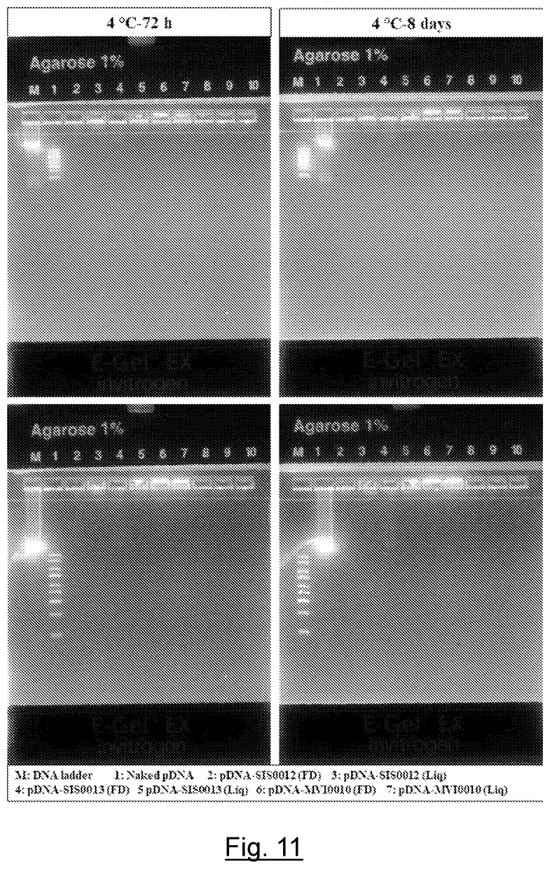

The agarose gel electrophoresis images of the pDNA-Biocourier formulations shortly after preparation and following storage at 4° C. for 8 days are presented in FIGS. 9 to 11. As observed, 6 h after storage at 4° C., only a small amount of pDNA passed through the gel with the freeze-dried forms of SIS0012 and SIS0013. However, the amount of unbound pDNA passing through the gel was low compared to the total amount of pDNA loaded on the Biocouriers indicating high binding efficiency. The liquid form of the formulations and also the freeze-dried form of MVI0010 did not release any pDNA into the gel indicating complete complexation of the loaded pDNA. The amount of pDNA passing through the gel for the freeze-dried form of SIS0012 and SIS0013 decreased after 24 h indicating some minor level of degradation of pDNA. From 48 h onwards, no traces of pDNA were observed for the freeze-dried SIS0012 whereas with freeze-dried SIS0013 there was still some traces of pDNA passing through the gel which continued until 8 days. This could be either the same unbound pDNA which was present from the beginning of the experiment or could be new pDNA dissociated from the complex. However, due to the very low amount of the DNA, it is hard to comment on its source with certainty other than to note that the vast majority of pDNA remained associated with the complex. The liquid form of all the formulations were able to retain all the loaded pDNA in the wells until 8 days indicating complete binding and stability at this storage condition.

### Conclusions

The liquid forms of all Biocourier formulations complex with the pDNA fully and were stable both at room temperature and at 4° C. for up to 8 days. However, the freeze-dried formulations, despite having a high binding efficiency, where unable to fully complex with all the loaded pDNA and a small amount of excess/unbound pDNA were found in these samples. Despite the lower binding efficiency of the freeze-dried formulations compared to their liquid counterparts, they were still stable both at room temperature and at 4° C. for up to 8 days.

### Example 4 Storage stability study with DNA or RNA

### Aim

To evaluate with agarose gel electrophoresis the stability of Biocourier formulations loaded with DNA or siRNA stored at different temperatures.

### Formulations Used

The following formulations (prepared as detailed above) were loaded with DNA, RNA or siRNA as noted
“Biocourier SIS0012”—formation containing undoped silicon particles
“Biocourier SIS0013”—formation containing boron doped silicon particles

### Methods

A. Preparation of the nucleic acid-Biocourier complexes

The nucleic acid stock solutions were prepared by dissolving the mRNA/siRNA/DNA powder in nuclease free water according to the table below:

| | | Volume of | Final |
|---|---|---|---|
| Type of | Amount of | nuclease free | concentration |
| nucleic acid | powder (mg) | water (μl) | (mg/ml) |
| siRNA | 3.33 (250 nMol) | 500 | 6.66 |
| mRNA from baker's | 5 | 1000 | 5 |
| yeast | | | |
| DNA from Herring | 3 | 1000 | 3 |
| testes Type XIV | | | |

The stock solutions were then diluted using nuclease free water to reach the required concentrations according to the table below:

| | | | | Final |
|---|---|---|---|---|
| Type of | Volume of stock | Volume of nuclease | Final volume | concentration |
| solution | solution (μl) | free water (μL) | (μl) | (ng/μl) |
| mRNA from | 4 | 196 | 200 | 100 |
| baker's yeast | | | | |
| (WS1) | | | | |
| mRNA from | 8 | 192 | 200 | 200 |
| baker's yeast | | | | |
| (WS2) | | | | |
| DNA from herring | 10 | 290 | 300 | 100 |
| testes Type XIV | | | | |
| siRNA | 2 | 198 | 200 | 66.5 |
| SIS0012 (WS1) | 4 | 196 | 200 | — |
| SIS0012 (WS2) | 10 | 190 | 200 | |
| SIS0012 (WS3) | 20 | 180 | 200 | |
| SIS0012 (WS4) | 40 | 160 | 200 | — |
| SIS0012- without | 40 | 160 | 200 | — |
| silicon | | | | |
| nanoparticles | | | | |
| SIS0013 | 40 | 160 | 200 | — |

The Biocourier stock solution was also diluted with nuclease free water to obtain the required concentrations for the complexation assay. The nucleic acid-Biocourier complexes were made by mixing the required amounts of the nucleic acid working solutions and Biocourier working solutions listed in the table below: and then incubated for 40 min at room temperature to ensure complete complexation:

| | Volume (μl/well) | | | | | |
|---|---|---|---|---|---|---|
| | DNA/RNA | SIS0012 | | Gel | | Amount of |
| Type of | working | working | | loading | | DNA/RNA |
| sample | solution | solution | | buffer | Total | (ng/well) |
| mRNA- | 2 (WS2) | 2.5 | (WS1) | 15.5 | 20 | 100 |
| SIS0012- 1 | | | | | | |
| mRNA- | 2 (WS2) | 5 | (WS2) | 13 | 20 | 200 |
| SIS0012- 2 | | | | | | |
| mRNA- | 2 (WS2) | 5 | (WS3) | 13 | 20 | 200 |
| SIS0012- 3 | | | | | | |
| mRNA- | 2 (WS2) | 5 | (WS4) | 13 | 20 | 200 |
| SIS0012- 4 | | | | | | |
| mRNA- | 2 (WS2) | 10 | (WS4) | 13 | 20 | 200 |

| | | | | | |
|---|---|---|---|---|---|
| SIS0012- 5 | | | | | |
| DNA- | 2 | 5 | 13 | 20 | 200 |
| SIS0012 | | | | | |
| siRNA- | 3 | 7.5 | 9.5 | 20 | 200 |
| SIS0012 | | | | | |
| siRNA- | 3 | 7.5 | 9.5 | 20 | 200 |
| SIS0013 | | | | | |
| siRNA | 3 | — | 16.5 | 20 | 200 |
| control | | | | | |
| mRNA | 2 | — | 18 | 20 | 200 |
| control | | | | | |
| DNA control | 2 | — | 18 | 20 | 200 |
| DNA ladder | 2 | — | 18 | 20 | 200 |

The prepared nucleic acid-Biocourier complexes were then subjected to analysis by gel electrophoresis.

### B. Agarose Gel Electrophoresis

The gel electrophoresis system used for these experiments consists of a gel electrophoresis device, precast agarose gels (with running buffer and electrode embedded within the gel) and a camera. The samples, controls (naked DNA or RNA), and DNA ladder were mixed with the required amount of the loading buffer to make a total volume of 20 μl according to the table above prior to loading onto the gel. Subsequently, the gel (1%) was inserted into the device chamber, loaded with the samples, controls and DNA ladder, and run for 7 min. The gel was transilluminated and imaged at 3 min and 7-8 min using the camera (the camera was mounted onto the device through the docking port available on the camera and the device). The same procedure was repeated at the designated time points (given in table above) following storage of the DNA-SIS0012, siRNA-SIS0012, and siRNA-SIS0013 at different storage conditions (room temperature (20° C.), 4° C., and −20° C.).

### Results

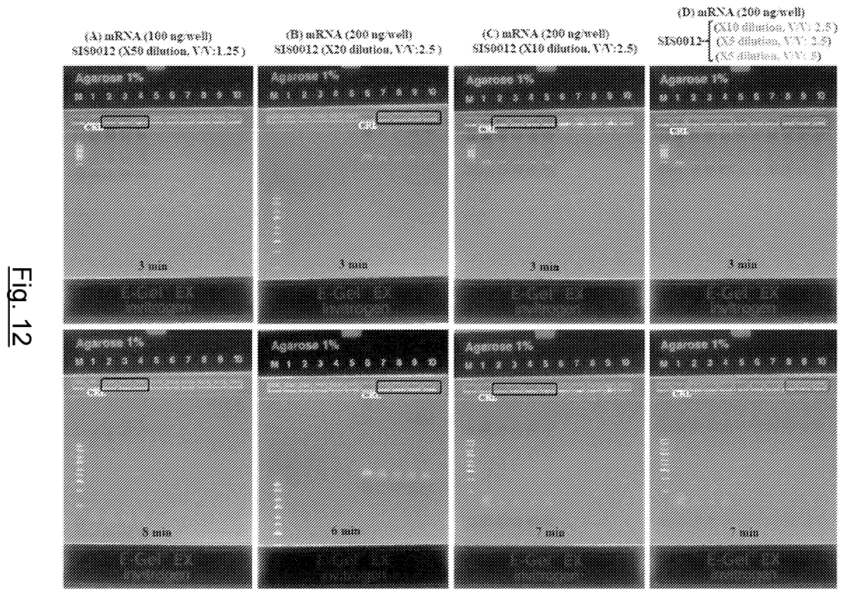

FIG. 12 shows the results from a gel retardation assay of the RNA from baker's yeast loaded onto SIS0012 at different concentrations of RNA, different concentrations of SIS0012 formulation and different volume ratios. It could be observed that 100 ng/well of the RNA which was recommended for this type of gel by the manufacturer resulted in very weak luminescence of the RNA whereas when the amount of RNA was increased to 200 ng/well a stronger signal was obtained. Therefore, the amount of nucleic acid was fixed at 200 ng/well for all future experiments. The SIS0012 formulation was mixed with RNA at different dilutions from the stock solution and different mixing ratios (v/v) to find out the minimum concentration of the SIS0012 solution and the lowest mixing ratio which could form complex with the RNA and inhibit its migration through the gel. It could be observed that 10× dilution from the SIS0012 stock solution at a mixing ratio of 2.5 partially blocks the migration of RNA through the gel as the strength of the luminescence (brightness of the bands) was reduced compared to the naked RNA which was used as a control and a complete block of the RNA migration was obtained when 5× diluted SIS0012 solution was mixed with RNA at the ratio of 2.5 or 4. Lower concentrations of the SIS0012 formulation or lower mixing ratios could not bind to RNA efficiently and could not inhibit its migration through the gel. Based on these findings, the 5× diluted SIS0012 solution and the mixing ratio of 2.5 (V/V) were selected for future experiments. Although the RNA used for these experiments could provide a proof of concept for the complexation assay, the purity of sample appeared to be low as the bands observed for the RNA were not sharp and not as strong as the DNA ladder. Moreover, the size of the RNA fragments was found to be very small (smaller than the smallest fragment of the DNA ladder which is 100 bp) indicating the presence of oligomers rather than full length mRNA.

Test to Evaluate the Complexation Efficiency and the Stability of DNA from Herring Testes Loaded onto SIS0012 Biocourier

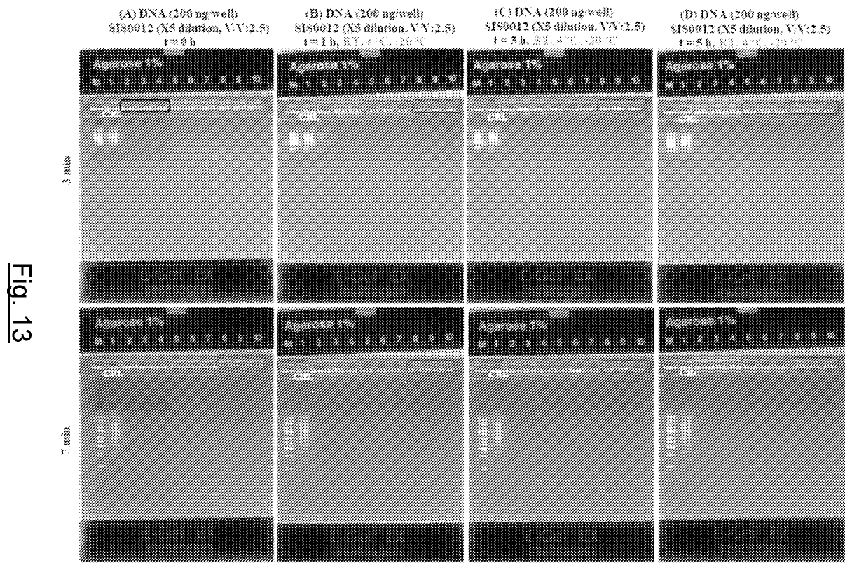

FIG. 13 shows the images from the agarose gel retardation assay of the DNA from herring testes loaded onto the SIS0012 formulation at the selected concentration and mixing ratio based on the previous test, immediately after complexation and until 5 h. The DNA-SIS0012 complexes were stored at three different conditions: room temperature, 4° C. and −20° C. It could be observed that the carrier entrapped the DNA efficiently and inhibited its migration through the gel (FIG. 13A). No signal from the DNA was observed in any of the gels which indicates no DNA was released by any of the three formulations (stored at different temperatures) within the first 5 h. Therefore, the experiments were further continued to study the stability of the DNA-SIS0012 complexes over a longer period of time. It is noteworthy that the DNA control (naked DNA) did not give a resolved band of specific size but rather showed a mixture of DNA fragments with different sizes. Nevertheless, since these experiments were merely aimed at assessing the complexation of DNA with the carrier and the stability of the corresponding complexes, for the purpose of these experiments there is no requirement for complete resolution of the DNA fragments or obtaining a single band of specific size which could be achieved by further treatment of the DNA by ultrasonication prior to using it for the complexation experiments.

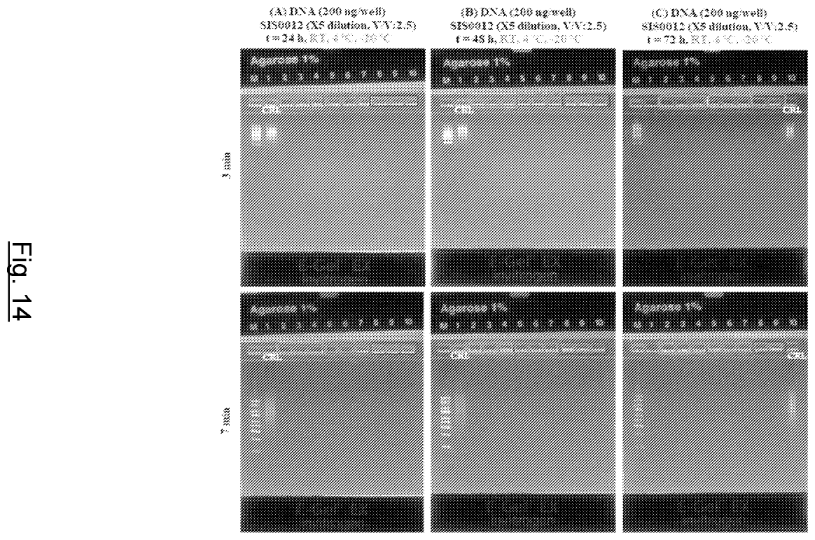

FIG. 14 shows the results from gel retardation assay of the DNA-SIS0012 complexes stored at different temperatures for up to 72 h. There was no evidence of DNA release from any of the three samples. Hence, it could be inferred from these observations that the DNA-SIS0012 complexes were stable at all the three different storage conditions for 72 h.

Test to Evaluate the Complexation Efficiency and the Stability of ADO-siRNA Loaded onto Biocourier-SIS0012

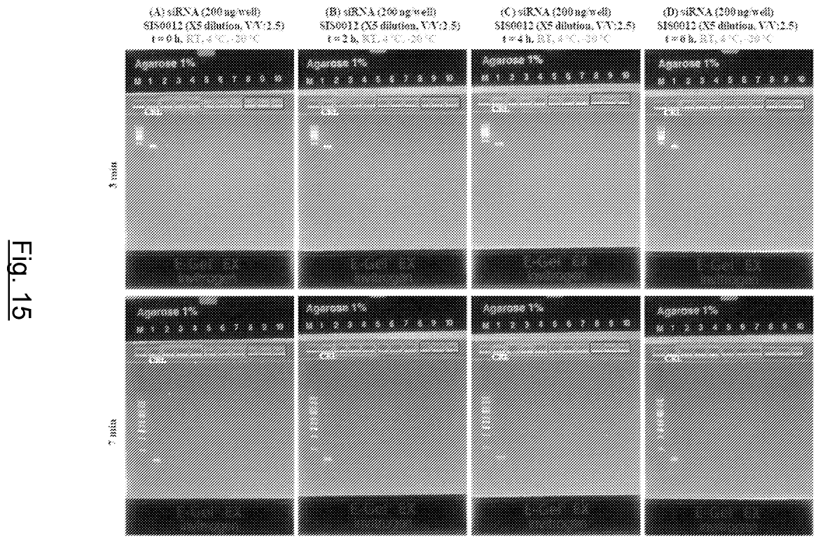

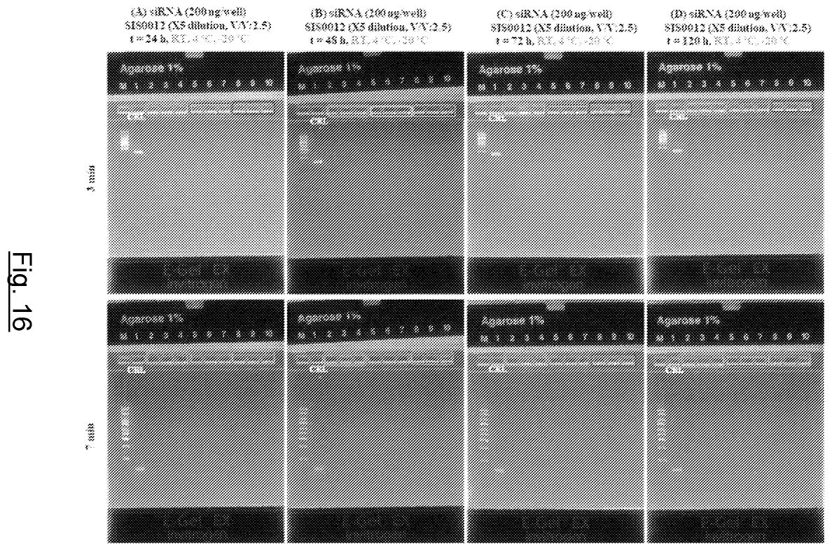

Following promising results from the DNA-SIS0012 experiments, the stability of ADO-siRNA-SIS0012 complexes produced by the same method (same concentration and mixing ratio) and stored at the same conditions (room temperature, 4° C. and −20° C.) was tested. The results are presented in FIGS. 15 and 16. As is evident from these figures, the SIS0012 formulation was able to entrap siRNA efficiently and to block its migration through the gel at all measured time points regardless of the storage condition which indicates stability of the siRNA-SIS0012 complexes in all three storage conditions for up to 120 h.

Test to Evaluate the Complexation Efficiency and the Stability of ADO-siRNA Loaded onto Biocourier-SIS0012 Without Silicon Nanoparticles

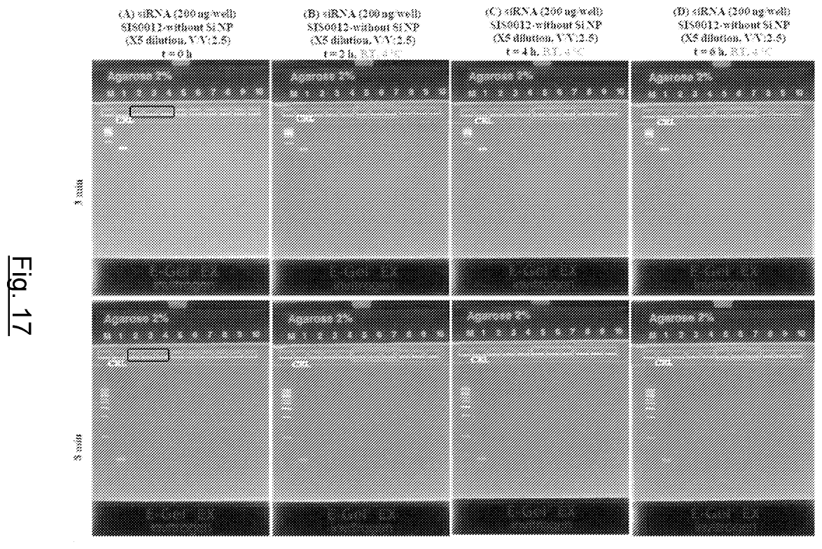

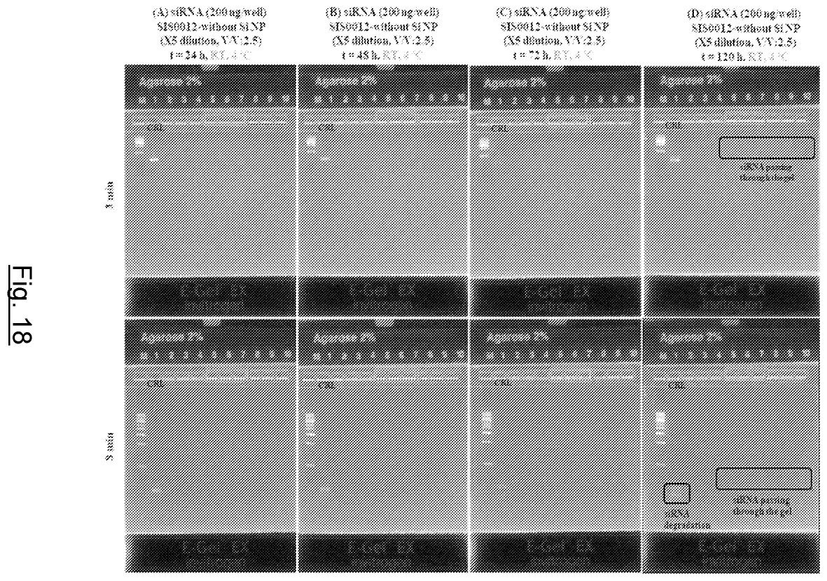

FIGS. 17 and 18 display the gel retardation assay results of ADO siRNA loaded onto SIS0012 formulation without silicon nanoparticles. The Biocourier was able to form complex with the siRNA and block its migration through the gel. Neither the sample stored at room temperature nor the sample stored at 4° C. released any amount of their associated siRNA at any of the measured time points until 6 h, as no bands for siRNA were observed. However, after 24 h some traces of siRNA were detected in the gel which gave rise to a very weak signal (indicating low amounts of siRNA) which was intensified at 120 h. Nevertheless, it should be noted that the samples at 72 h and 120 h were run without any gel loading buffer and only diluted with water prior to loading onto the gel. Therefore, the intensity of the observed signal would be less than the signal observed when the samples are diluted using the gel loading buffer. Another observation which is noteworthy is the presence of two bands for the control siRNA indicating degradation of the siRNA. The siRNA solution used as control in these experiments (66 μg/ml) was stored at −20° C. and defrosted prior to commencing the experiment each time. Hence, it is supposed that the siRNA degradation is likely a result of repeated freezing-thawing cycles in a short time period.

Test to Evaluate the Complexation Efficiency and the Stability of ADO-siRNA Loaded onto Biocouriers-SIS0013

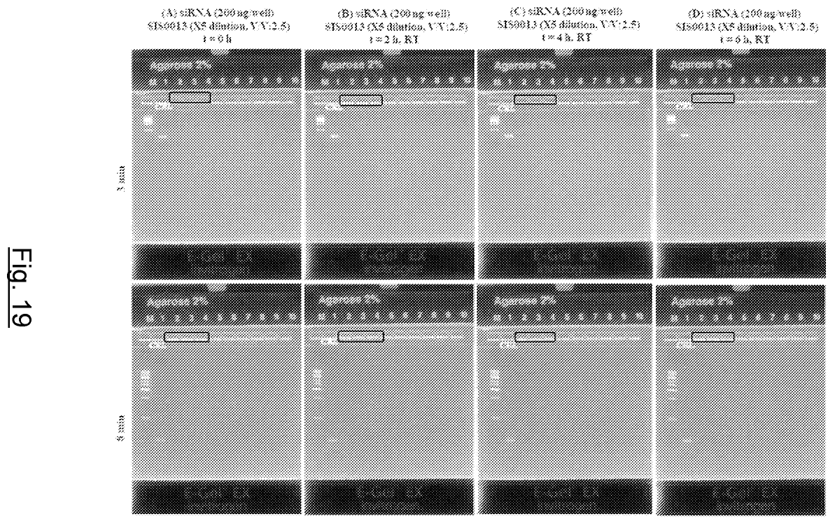

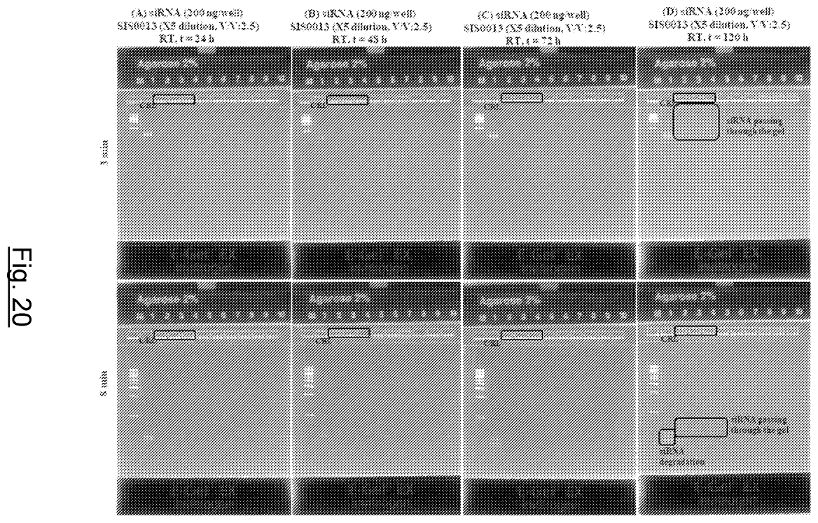

FIGS. 19 and 20 show the gel retardation assays performed on ADO-siRNA loaded onto SIS0013 formulation immediately after complexation and until 6 h after storage at room temperature. The Biocouriers were able to entrap the siRNA and although some traces of siRNA could be observed throughout the gel from the beginning of the experiment, the signal was not significant before 120 h. At 120 h post storage, the signal was stronger indicting increased release of siRNA from the complexes. However, even at this time point the signal was markedly weaker compared to the signal from naked siRNA indicating very low amounts of siRNA passing through the gel. It should also be noted that the samples at 72 h and 120 h were run without any gel loading buffer and only diluted with water prior to loading onto the gel. Therefore, the intensity of the observed signal would be less than the signal observed when the samples are diluted using the gel loading buffer. Similar to what is illustrated in FIG. 16, the naked siRNA exhibited two bands indicating degradation of the siRNA due to multiple freezing-thawing cycles.

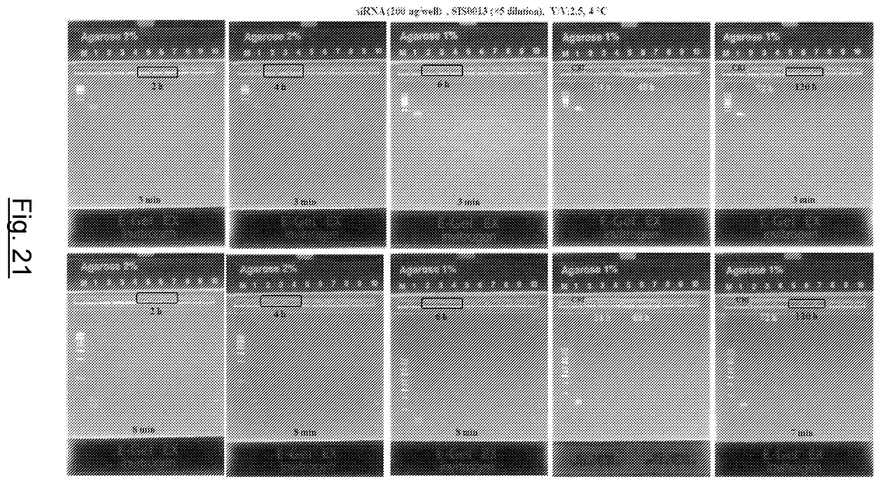

Unlike storage at room temperature, siRNA-SIS0013 complexes stored at 4° C. remained stable for up to 120 h as indicated by lack of siRNA migration through the gel (FIG. 21).

### Conclusion

The gel retardation assay developed using the reported experimental parameters is efficient in detecting the complexation of the nucleic acids with the Biocourier and in testing the stability of the formed complexes over time using very small volumes (2-3 μl/well) and low concentrations (100 ng/μl) of nucleic acids and could be applied to different types of nucleic acids (DNA, mRNA, siRNA). The optimum complexation condition for the nucleic acid and Biocourier was found to be at the mixing ratio of 2.5 V/V, with 5× diluted Biocourier solution and DNA/mRNA (100 μg/ml) or siRNA (66.5 μg/ml). The Biocourier formulation SIS0012 was able to retain its associated siRNA for up to 120 h whereas the SIS0012 formulation without silicon nanoparticles started releasing the siRNA after 24 h and after 120 h the amount of siRNA released through the gel was significant. The SIS0013 formulation released small amounts of siRNA into the gel even at the starting point suggesting lower entrapment efficiency compared to SIS0012 and after 120 h the amount of siRNA passing through the gel became significant. However, SIS0013-siRNA complexes were stable at 4° C. for up to 120 h demonstrating superior storage stability when the silicon particles comprise boron-doped silicon.

### Example 5—Stabilisation of Alkaline Phosphatase by SIS0012 (undoped Si) vs. SIS0013 (boron doped Si; 5×1018 boron atoms/cm³)

Alkaline phosphatase is an enzyme that exists in various forms, catalyses the degradation of various proteins, and may be found in all tissues in the human body. It is mostly concentrated in the bones, kidneys, liver, intestines, and placenta. It contributes, inter alia, to: protection of the intestinal tract against bacteria; digestive function; degradation of fats and vitamin B; and bone formation. Alkaline phosphatase exhibits a loss of activity at low pH and at high temperature.

Alkaline phosphatase activity can be monitored by measuring changes in the concentration, for which UV-Vis absorbance is a proxy, of one or more of its substrates or products in an in vitro assay. For example, the concentration of the substrate 4-nitrophenyl phosphatase (PNPP) the structure of which is set out below, may be monitored to track the following reaction:

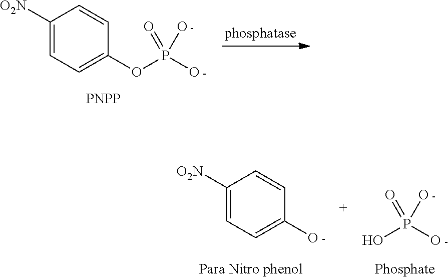

### Materials, Methods and Results

Alkaline phosphatase, isolated from bovine intestine and supplied as a recombinant enzyme of 56 kD, expressed in the yeast, Pichia Pastoris, was obtained from Sigma Aldrich/Merck (The Old Brickyard, New Rd, Gillingham, Dorset, SP8 4XT). An aqueous stock solution of alkaline phosphatase (ALP) was prepared at a concentration of 1 U/ml. 1 U (μmol/min) is defined as the amount of ALP that catalyzes the conversion of one micromole of PNPP per minute at 37° C. and pH 7.4.

A 20 mM solution of 4-nitrophenyl phosphatase (PNPP) was prepared using Tris Buffer at pH 7.4 (100 mM/L).

SIS0012 (undoped Si) and SIS0013 (boron-doped Si; 5×10¹⁸ boron atoms/cm³) samples were provided according to the protocol of Example 2.

ALP solutions were prepared at the following concentrations:

0.1, 0.5, 1, 5, 10, 50, and 100 mU/ml from the 1U/ml stock solution, in 15ml test tubes.

These were then mixed, in Eppendorf tubes, with the prepared 20 mM solution of PNPP Tris buffer.

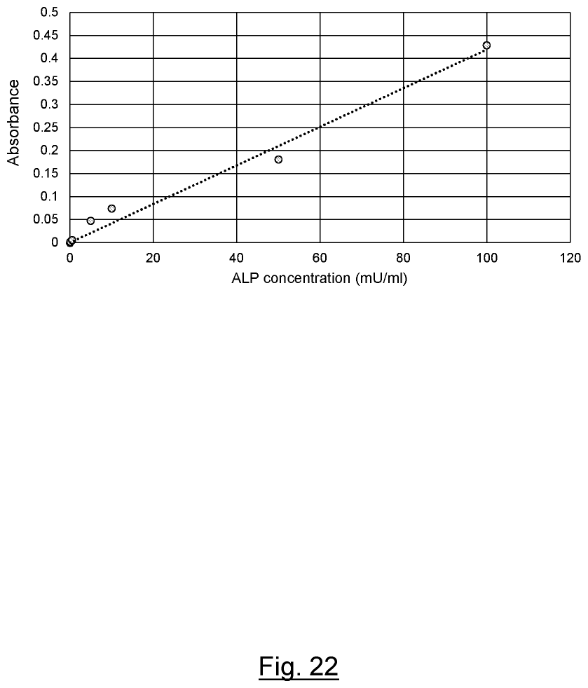

The tubes were incubated in a water bath at 37° C. for 30 minutes, following which, UV-Vis absorbance measurements were made at 405 nm as shown in FIG. 22.

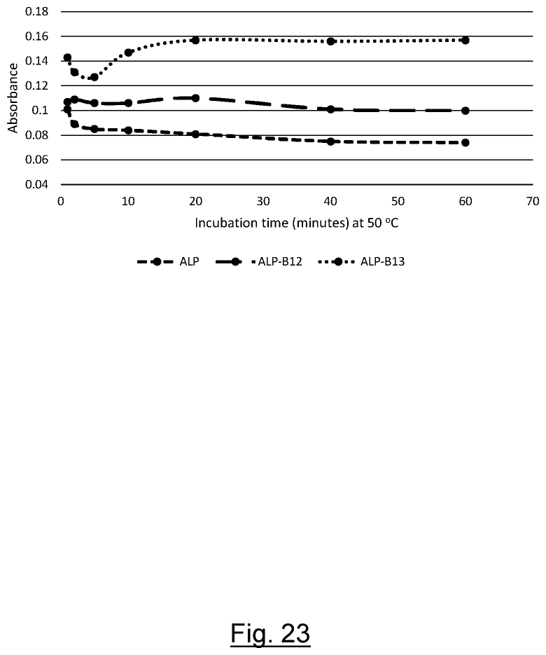

ALP was loaded onto SIS0012 and SIS0013 as follows.
1.3 sets of 8 Eppendorf tubes were prepared:
A 8 Eppendorf tubes were prepared, each containing 50 μL of ALP (50mU/ml) and 500 μL of SIS0012 (undoped Si). After adding these components to the tubes, they were mixed, vortexed and refrigerated overnight.
B 8 Eppendorf tubes were prepared, each containing 50 μL of ALP (50mU/ml) and 500 μL of SIS0013 (boron doped Si). After adding these components to the tubes, they were mixed, vortexed and refrigerated overnight.
C 8 Eppendorf tubes were prepared, each containing 50 μL of ALP (50mU/ml) and 500 μL of Tris buffer. After adding these components to the tubes, they were mixed, vortexed and refrigerated overnight.
2. Following their preparation, all Eppendorf tubes were placed in a water bath at 50° C. Each of the eight tubes, in each of the three sets of tubes A to C, was removed from the water bath after 1, 2, 5, 10, 20, 40 or 60 minutes.
3. 300 μL of PNPP was then added to all Eppendorf tubes in all sets A to C. The tubes were mixed and vortexed. They were then placed in a water bath at 37° C. for 30 minutes, during which time dephosphorylation of the PNPP occurred.
4. Following this, UV-Vis analysis was carried out on all samples in all sets A to C (at 405 nm). The results are shown in FIG. 23.

### Discussion

Surprisingly, the activity of ALP loaded on SIS0013 (boron doped Si) was approximately 20-30% higher than the activity of ALP loaded on SIS0012. It is thought that this is due to improved protection of ALP by doped Si.

Free ALP showed significant degradation compared to both SIS0012 and SIS0013. The activity of free ALP decreased with increasing incubation time at 50° C. On the other hand, the activity of ALP loaded onto SIS0012 and SIS0013 remained relatively constant regardless of incubation time. It is thought that free ALP denatures over time at 50° C.; while ALP loaded onto SIS0012 and SIS0013, particularly SIS0013, is protected from denaturing and remains stable.

### Example 6—Complexation of Further Silicon-Containing Formulations with mRNA or siRNA

Alternatives were investigated to replace cationic lipids, such as DOTAP used in SIS0012 and SIS0013. One such alternative is lipopeptides. These are amphiphiles that consist of a lipid chain (generally, 12-18 carbon-atoms long) conjugated to a peptide sequence (generally, of 3-20 amino acid residues). A particular example of such lipopeptides is palmitoyl pentapeptide-4 (abbreviated as PAL-KTTKS):

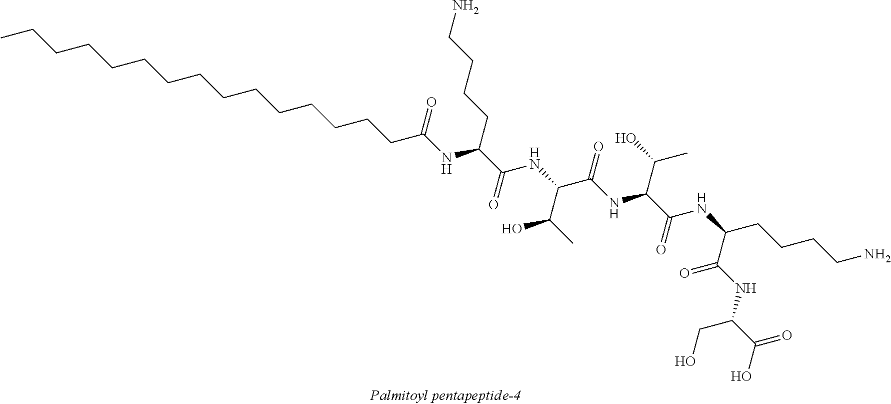 [SMILES: CCCCCCCCCCCCCCCC(=O)N[C@@H](CCCCN)C(=O)N[C@H](C(=O)N[C@H](C(=O)N[C@@H](CCCCN)C(=O)N[C@@H](CO)C(=O)O)[C@@H](C)O)[C@@H](C)O]

PAL-KTTKS is thought to be a good candidate for an alternative to cationic lipids, due to its cationic lysine residues, which assist binding to negatively charged RNA.

Other candidates investigated with SIS0012 and SIS0013 include NAD, tyrosine (TYR) and quercetin (QUE). With these ligands, it is thought that organ-specific uptake of RNA may be enhanced, and that cell internalisation and targeting may be improved. Moreover, they can assist in providing a positively charged environment for binding of negatively charged RNA.

Nicotinamide adenine dinucleotide (NAD) is a coenzyme. In its oxidised form, NAD+, it has the following structure:

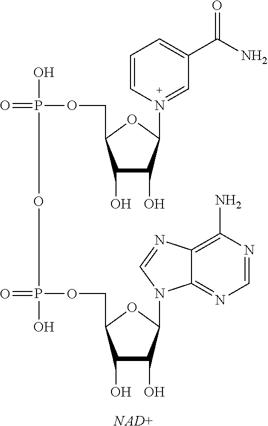

Tyrosine is a naturally occurring amino acid, having the following structure at physiological pH (pH 7.4):

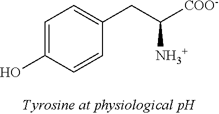

Quercetin is a flavanol, having the following structure:

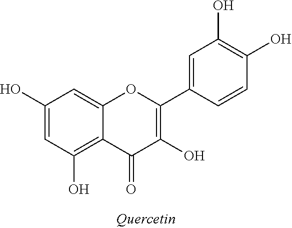 [SMILES: O=c1c(O)c(-c2ccc(O)c(O)c2)oc2cc(O)cc(O)c12]

NAD, TYR and QUE—investigation of addition to, or replacement of, DOTAP in SIS0012 and SIS0013

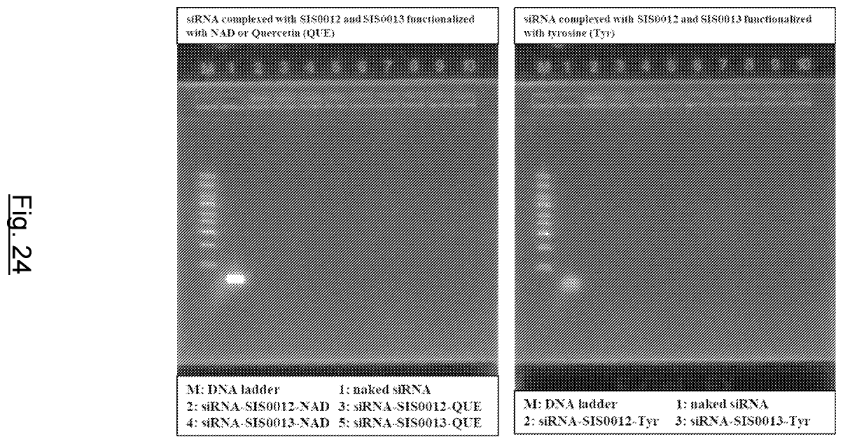

Modified SIS0012 and SIS0013 compositions were prepared by adding, to the usual compositions as set out above (i.e., in addition to DOTAP), 0.2 mg of NAD, TYR and QUE. Complexation with siRNA was then assessed. FIG. 24 shows gel electrophoresis results that indicate siRNA was successfully and fully bound in these formulations.

Dynamic light scattering measurements were also performed, using a Zetasizer (obtainable from Malvern Instruments) to assess size and charge, both prior to and following the formation of siRNA complexes, as shown in the table below. As the table indicates, size increases were observed after siRNA complexation, along with a 10-15 mV drop in surface charge.

| | Size (nm) | | PDI | | Zeta Potential (mV) | |
|---|---|---|---|---|---|---|
| Sample | Before | After | Before | After | Before | After |
| Code | Complexation | Complexation | Complexation | Complexation | Complexation | Complexation |
| SIS0012- | 119.0 ± 5.5 | 216.9 ± 1.5 | 0.22 ± 0.04 | 0.12 ± 0.01 | 58.3 ± 1.1 | 47.1 ± 0.5 |
| Empty | | | | | | |
| SIS0012 - | 90.9 ± 1.3 | 186.0 ± 2.0 | 0.09 ± 0.02 | 0.25 ± 0.01 | 60.6 ± 1.0 | 42.7 ± 0.2 |
| NAD | | | | | | |
| SIS0012 - | 91.6 ± 0.3 | 217.4 ± 2.5 | 0.07 ± 0.02 | 0.39 ± 0.01 | 63.2 ± 4.5 | 41.6 ± 0.2 |
| QUE | | | | | | |
| SIS0012 - | 77.7 ± 0.5 | 334.0 ± 19.2 | 0.09 ± 0.01 | 0.45 ± 0.04 | 67.5 ± 1.1 | 44.8 ± 0.3 |
| TYR | | | | | | |
| SIS0013- | 100.0 ± 0.4 | 165.8 ± 3.3 | 0.16 ± 0.03 | 0.19 ± 0.01 | 55.1 ± 1.4 | 46.9 ± 0.7 |
| Empty | | | | | | |
| SIS0013 - | 104.8 ± 1.7 | 178.3 ± 1.5 | 0.09 ± 0.01 | 0.18 ± 0.01 | 58.5 ± 2.2 | 41.8 ± 0.3 |
| NAD | | | | | | |
| SIS0013 - | 99.8 ± 0.3 | 175.2 ± 0.9 | 0.05 ± 0.01 | 0.25 ± 0.01 | 60.3 ± 0.8 | 43.3 ± 0.5 |
| QUE | | | | | | |
| SIS0013 - | 94.2 ± 0.9 | 160.7 ± 1.1 | 0.09 ± 0.01 | 0.22 ± 0.01 | 64.6 ± 0.4 | 48.6 ± 0.4 |
| TYR | | | | | | |

The replacement of DOTAP by NAD, TYR or QUE (rather than simply addition of NAD, TYR or QUE to DOTAP-containing compositions) was then investigated.

For this study, DPPC and DOPE were selected for use alongside NAD, TYR or QUE, as DPPC and DOPE are zwitterionic lipids that play no significant role in surface charge at neutral pH.

Thus, DPPC/DOPE formulations were prepared with (i) 0.2 mg and (ii) 1 mg of NAD, TYR and QUE. The formulations are set out in the tables below.

TABLE
Composition of DPPC/DOPE LNP Biocourier functionalised
with beta nicotinamide adenine dinucleotide (NAD).

| | | | Hydrating solution | | | | |
|---|---|---|---|---|---|---|---|
| | Lipid phase | | Activated | | | | |
| Formulation | DPPC | DOPE | siNPs (30 nm) | Glycine | Trehalose | NAD | NFW |
| DPPC/DOPE- | 7.75 mg | 8.25 mg | 1 mg | 0.5 mg | 1 mg | 0.2 mg | Up to |
| NAD-0.2 mg | | | | | | | 10 mL |
| DPPC/DOPE- | 7.75 mg | 8.25 mg | 1 mg | 0.5 mg | 1 mg | 1 mg | Up to |
| NAD-1 mg | | | | | | | 10 mL |

TABLE
Composition of DPPC/DOPE LNP Biocourier functionalised with Quercetin (QUE).

| | | | | Hydrating solution | | | |
|---|---|---|---|---|---|---|---|
| | Lipid phase | | | Activated | | | |
| Formulation | DPPC | DOPE | Quercetin | siNPs (30 nm) | Glycine | Trehalose | NFW |
| DPPC/DOPE- | 7.75 mg | 8.25 mg | 0.2 mg | 1 mg | 0.5 mg | 1 mg | Up to |
| QUE-0.2 mg | | | | | | | 10 mL |
| DPPC/DOPE- | 7.75 mg | 8.25 mg | 1 mg | 1 mg | 0.5 mg | 1 mg | Up to |
| QUE-1 mg | | | | | | | 10 mL |

TABLE
Composition of DPPC/DOPE LNP Biocourier SIS0012
functionalised with Tyrosine (TYR)

| | | | Hydrating solution | | | | |
|---|---|---|---|---|---|---|---|
| | Lipid phase | | Activated | | | | |
| Formulation | DOTAP | DOPE | SiNPs (30 nm) | Glycine | Trehalose | Tyrosine | NFW |
| DPPC/DOPE- | 7.75 mg | 8.25 mg | 1 mg | 0.5 mg | 1 mg | 0.2/1 mg | Up to |
| TYR-0.2 mg | | | | | | | 10 mL |
| DPPC/DOPE- | 7.75 mg | 8.25 mg | 1 mg | 0.5 mg | 1 mg | 0.2/1 mg | Up to |
| TYR-1 mg | | | | | | | 10 mL |

Zetasizer measurements were obtained, revealing negative zeta potentials across all formulations, irrespective of the amount of NAD, TYR or QUE; as shown in the table below.

TABLE
DPPC/DOPE LNPs functionalised with 0.2
mg and 1 mg of NAD, TYR and QUE.

| Sample | Size | PDI | Zeta - Empty |
|---|---|---|---|
| DPPC/DOPE-NAD - 0.2 | 78.46 ± 2.65 | 0.18 ± 0.04 | −20.76 ± 0.68 |
| mg | | | |
| DPPC/DOPE-NAD - 1 mg | 88.46 ± 1.22 | 0.16 ± 0.05 | −25.75 ± 0.57 |
| DPPC/DOPE-QUE - 0.2 | 86.0 ± 0.63 | 0.17 ± 0.02 | −25.81 ± 0.82 |
| mg | | | |
| DPPC/DOPE-QUE - 1 mg | 78.35 ± 2.19 | 0.18 ± 0.02 | −20.2 ± 1.43 |
| DPPC/DOPE-TYR - 0.2 | 84.16 ± 2.33 | 0.19 ± 0.03 | −24.99 ± 0.44 |
| mg | | | |
| DPPC/DOPE-TYR - 1 mg | 83.21 ± 2.57 | 0.16 ± 0.03 | −18.81 ± 0.46 |

Lipopeptides—investigation of replacement of DOTAP in SIS0012 and SIS0013

Also known as peptide amphiphiles (PA), lipopeptides were explored as another alternative to DOTAP.

It is thought that lipopeptides may offer a solution to the problem of replacing, or reducing the amount of, cationic lipids such as DOTAP, in transfection compositions. Lipopeptides consist of an alkyl chain conjugated to a peptide sequence. It is thought that their alkyl chain may be assimilated in lipid bilayers, while the surface of the bilayer is decorated with the peptide moiety.

An exemplary PA is the molecule palmitoyl pentapeptide-4 (abbreviated as PAL-KTTKS, see above). The two cationic lysine residues may perform a similar function to cationic lipid, such as DOTAP, exhibiting an electrostatic interaction with negatively charged RNA.

DPPC and DOPE were selected as neutral lipids to formulate with PAL-KTTKS. Various formulations were prepared with different silicon nanoparticles or without silicon at all; and with pH 4 buffer (to investigate the effect of pH).

Full details of the DPPC, DOPE and PAL-KTTKS-containing formulations are provided in the table below.

TABLE
Various formulation compositions of DPPC, DOPE and Pal-KTTKS with silicon
nanoparticles, boron-doped silicon and boric acid/silicon nanoparticles.
Formulation counterparts without silicon nanoparticles were also formulated
as controls. All formulations were prepared up to 10 ml final volume.

| | Lipids | | | | | | Total |
|---|---|---|---|---|---|---|---|
| Sample | | Lip- | | Silicon-Trehalose-Glycine | | | lipid |
| Sample | DPPC | Peptide | DOPE | Silicon | Trehalose | Glycine | amount |
| 1- KTTKS- | 7.75 | 8.25 | 8.25 | 1 (Classic | 1 | 0.5 | 24.25 |
| BC | | | | AE Si) | | | |
| 2- KTTKS- | 8.25 | 7.75 | — | 1 (Classic | 1 | 0.5 | 16 |
| BC | | | | AE Si) | | | |
| 3- KTTKS- | 3.875 | 3.875 | 8.25 | 1 (Classic | 1 | 0.5 | 16 |
| BC | | | | AE Si) | | | |
| 4- KTTKS- | 7.75 | 8.25 | 8.25 | 1 (Boron | 1 | 0.5 | 24.25 |
| BC | | | | doped Si) | | | |
| 5- KTTKS- | 7.75 | 8.25 | 8.25 | 1 (Boric | 1 | 0.5 | 24.25 |
| BC | | | | acid Si) | | | |
| 6- KTTKS- | 7.75 | 8.25 | 8.25 | 1 (Classic | 1 | 0.5 | 24.25 |
| BC pH 4 | | | | AE Si) | | | |
| buffer | | | | | | | |
| 7- KTTKS- | 7.75 | 8.25 | 8.25 | — | — | — | 24.25 |
| BC pH 4 | | | | | | | |
| buffer | | | | | | | |
| 8- KTTKS- | 7.75 | 8.25 | 8.25 | — | — | — | 24.25 |
| BC(Nuclease | | | | | | | |
| free water) | | | | | | | |

All 8 formulations had positively charged surfaces, with zeta potentials (measured with a Zetasizer, obtainable from Malvern Instruments) provided in the table below.

Based on these results, it is thought that during assembly of the lipid film, PAL-KTTKS is arranges itself in the lipid bilayer with the peptide portion exposed on the nanoparticle surface. Furthermore, the lysine residues on that surface contribute to a positively charged formulation.

TABLE
Zeta potential measurements for all
8 DPPC/DOPE/PAL-KTTKS formulations.

| Sample | Zeta - Empty |
|---|---|
| 1- KTTKS-BC | 60.09 ± 0.84 |
| 2- KTTKS-BC | 48.48 ± 2.54 |
| 3- KTTKS-BC | 37.24 ± 0.63 |
| 4- KTTKS-BC | 54.38 ± 2.11 |
| 5- KTTKS-BC | 55.43 ± 1.11 |
| 6- KTTKS-BC pH 4 buffer | 59.44 ± 0.81 |
| 7- KTTKS-BC pH 4 buffer | 55.51 ± 0.39 |
| 8- KTTKS-BC(Nuclease | 53.11 ± 0.39 |
| free water) | |

All formulations were assessed for their ability to electrostatically bind to siRNA and mRNA.

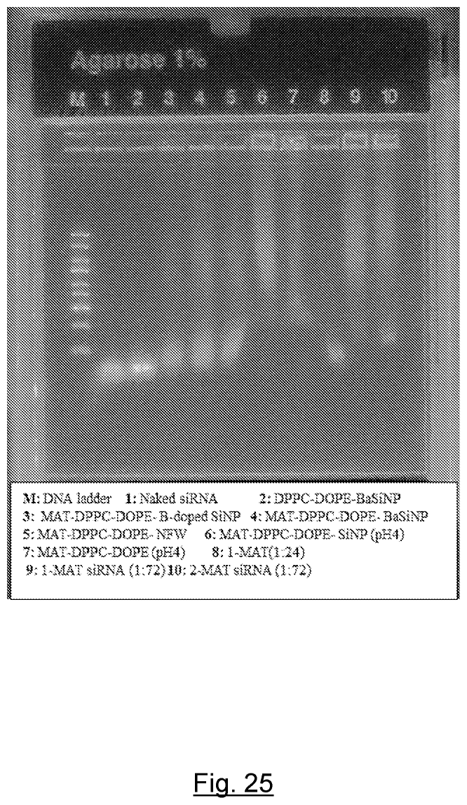

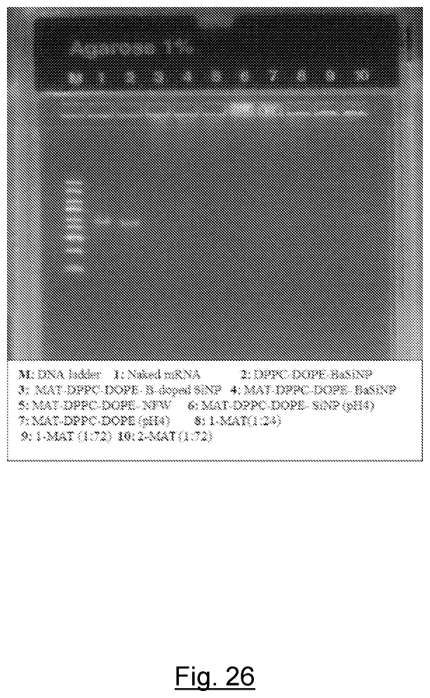

Gel electrophoresis analysis was performed. Full complexation was not observed for siRNA. However, full complexation was observed for mRNA. FIG. 25 shows the gel electrophoresis results for siRNA; FIG. 26 shows the gel electrophoresis results for mRNA.

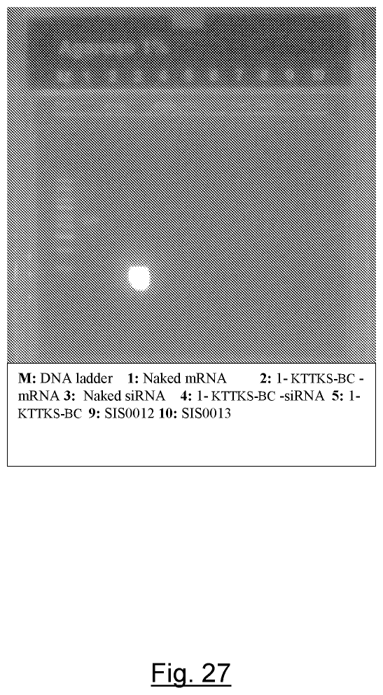

To address the partial complexation of siRNA with DPPC/DOPE/PAL-KTTKS, an alternative loading method was adopted. FIG. 27 shows gel electrophoresis of complexes after the alternative loading method was used, indicating successful complete complexation of siRNA. (The bright dot in lane 3 of FIG. 27 is an artefact of the imaging device.)

In the alternative loading method, compared to the protocol described hereinabove, the following steps were adopted:
1. A thin lipid film was prepared by dissolving DPPC, DOPE and Pal-KTTKS in methanol and evaporating using a rotary evaporator.
2. The lipid film was rehydrated with a suspension containing activated silicon (SIS0012) or activated boron-doped silicon (SIS0013) with trehalose and glycine as well as either siRNA or mRNA. Reydration was performed at 40° C. for 10 mins to ensure no lipid-silicon film remained on the walls of the round bottomed rot-evap flask.

Lipopeptides are highly versatile molecules: they may be fine-tuned, by altering their alkyl chain and/or their peptide sequence. It is thought that customisation of the peptide may enhance cell and/or tissue targeting. In the field of gene therapy, customisation of the peptide may enahnce electrostatic interactions with nucleic acids, such as RNA, particularly mRNA. As an example, PAL-KTTKS, when formulated with DPPC and DOPE, resulted in a positively charged surface, as confirmed by zeta potential.

Lipopeptides, being amphiphilic molecules, have very similar properties to surfactants, which can self-assemble to form micelles: it is thought that this is due at least in part to the alkyl chain being amenable to hydrophobic interactions, whilst the peptide sequence can form inter-molecular hydrogen bonding. Phospholipids, such as DPPC and DOPE, are also able to self-assemble into liposomes. Thus, when incorporating PAs, of which PAL-KTTKS is a representative example (although other lipopeptides may be used) the alkyl chain is able to form hydrophobic interactions with DPPC and DOPE leading to liposomal structures.

Meanwhile, the silicon nanoparticles provide structural stability for the complex as a whole, and are able to interact with the lipid and other ligands, such as lipopeptide, NAD, QUE or TYR, via non-covalent (electrostatic) interactions, thus promoting long-term stability and binding of nucleic acid.

1. A pharmaceutical composition comprising particles which comprise hydrolysable doped silicon and one or more lipids complexed with an active pharmaceutical ingredient, wherein the particles are doped at a level of at least 1×10¹⁶ dopant atoms per cm³.

2. The pharmaceutical composition according to claim 1, wherein the hydrolysable doped silicon particles are doped at a level of up to 1×10²⁰ dopant atoms per cm³.

3. The pharmaceutical composition according to claim 1, wherein the active pharmaceutical ingredient is a nucleic acid.

4. The pharmaceutical composition according to claim 3, wherein the nucleic acid is RNA.

5. The pharmaceutical composition according to claim 4, wherein the nucleic acid is small interfering RNA (siRNA) or small activating RNA (saRNA) or small hairpin RNA (shRNA).

6. The pharmaceutical composition according to claim 4, wherein the nucleic acid is messenger RNA (mRNA).

7. The pharmaceutical composition according to claim 6, wherein the mRNA encodes a protein of a pathogenic organism.

8. The pharmaceutical composition according to claim 1, wherein the pharmaceutical composition is a vaccine composition.

9. The pharmaceutical composition according to claim 1, wherein the pharmaceutical composition further comprises an amino acid.

10. The pharmaceutical composition according to claim 1, wherein the pharmaceutical composition further comprises a non-reducing disaccharide.

11. The pharmaceutical composition according to claim 1, wherein the doped silicon particles comprise boron-doped silicon.

12. The pharmaceutical composition according to claim 1, wherein the doped silicon particles comprise phosphorus-doped silicon.

13-14. (canceled)

15. The pharmaceutical composition according to claim 1, wherein the one or more lipids comprises an ionisable lipid.

16. The pharmaceutical composition according to claim 1, wherein the one or more lipids comprises a cationic lipid.

17. The pharmaceutical composition according to claim 16, wherein the one or more lipids comprise DOTAP.

18-20. (canceled)

21. The pharmaceutical composition according to claim 1, wherein the one or more lipids comprise one or more lipidated oligopeptides.

22. A method of enhancing the efficiency of a pharmaceutical composition comprising an active pharmaceutical ingredient, said method comprising contacting the active pharmaceutical ingredient with hydrolysable doped silicon particles, wherein the hydrolysable doped silicon particles are doped at a level of at least 1×10¹⁶ dopant atoms per cm³.

23-31. (canceled)

32. A method of treating or preventing a disease or disorder comprising administering to a subject in need thereof a pharmaceutical composition according to claim 1.

33. (canceled)

34. A method of increasing the storage stability of an active pharmaceutical ingredient comprising contacting the active pharmaceutical ingredient with hydrolysable doped silicon particles and one or more lipids, wherein the hydrolysable doped silicon particles are doped at a level of up to 1×10¹⁶ dopant atoms per cm³.

35-38. (canceled)

39. A method of targeting an active pharmaceutical ingredient to a cell or tissue, comprising administering to a subject in need thereof a pharmaceutical composition according to claim 1.
